(19) United States
(12) Patent Application Publication
(10) Pub. No.: US 2024/0165385 A1
(43) Pub. Date: May 23, 2024

(54) INTRAOSSEOUS INFUSION PORTS AND METHODS OF USE

(71) Applicant: PAVmed, Inc., New York, NY (US)

(72) Inventors: Lishan Aklog, New York, NY (US); Brian J. deGuzman, New York, NY (US); Richard Yazbeck, New York, NY (US); Michael Boutillette, New York, NY (US); Taylor Bensel, New York, NY (US)

(21) Appl. No.: 18/282,958

(22) PCT Filed: Apr. 19, 2022
(86) PCT No.: PCT/US2022/025291

§ 371 Date: Sep. 19, 2023

(51) Int. Cl. A61M 39/02

(52) U.S. Cl. CPC A61M 39/02

(57) ABSTRACT

An intraosseous port device includes a body having a proximal portion configured and arranged for subcutaneous placement and having a first channel, and a distal portion designed for intraosseous placement at a site of interest and having a second channel in fluid communication with the first channel to define a pathway from the proximal portion to the site of interest, a penetrable seal occluding at least a portion of the first channel, and a retention ring coupled to at least one of the body and the penetrable seal.

### CROSS-REFERENCE TO RELATED APPLICATION

This application claims priority to, and the benefit of, U.S. Provisional Application No. 63/178,783, filed Apr. 23, 2021, for all subject matter common to both applications. The disclosure of said provisional application is hereby incorporated by reference in its entirety.

### FIELD OF THE INVENTION

The present invention relates to an intraosseous infusion port suitable for medical use. In particular, the present invention relates to an intraosseous infusion port suitable for directing a medical device to a site of interest with minimal efforts providing smooth insertion of a medical device while placed within tissue.

### BACKGROUND

Vascular access devices (VADs) are widely used to deliver a wide variety of substances, including fluids, medications including chemotherapy, blood products and total parental nutrients. Transcutaneous vascular access devices include standard central venous catheters (CVC), tunneled central venous catheters and peripherally inserted central venous catheters (PICC). These catheters pass through the skin, enter a vein, and terminate in a central venous location. They can have one or more lumens and corresponding hubs. Transcutaneous VADs can be left at the insertion site for weeks or more, and as such require regular flushes with saline or an anti-coagulant solution to protect against thrombus formation and occlusion. Other types of VADs such as totally implantable VADs are used in patients who require access for weeks to months. The implantable VADs typically have a metal or plastic port that is implanted into the subcutaneous tissue and anchored to the fascial tissue along with a catheter portion that enters a vein. The implantable VADs also require regular flushes with anti-coagulant solution, typically concentrated heparin to prevent thrombus formation and occlusion.

Currently available VADs bear significant risk (about 10%) of introducing infection to a blood stream, which can lead to serious costly complications such as bacteremia, sepsis, or even death. Furthermore, because they are in constant contact with the blood stream, the VADs require regular flushes to clear stagnant blood and prevent thrombus formation and occlusion. Even with regular flushes occlusions occur in approximately 30% of patients, requiring treatment with thrombolytic agents or device removal and reinsertion of a new device, which are costly, can interfere with patient care and result in complications. In addition, most VADs require radiologic (chest radiograph or fluoroscopy) confirmation of proper positioning in a central venous location and must be carefully handled by trained clinicians. A trained clinician is required because the introduction of an even modest amount of air into the device can lead to catastrophic air embolism, which can be fatal. Still furthermore, VADs must generally terminate in, or at least in the vicinity of, the right atrium. Repeated instrumentation in chronically ill patients, such as hemodialysis patients, can lead to venous fibrosis, stenosis, and occlusion, which can lead to significant morbidity and can be a formidable challenge in patients who still require vascular access.

Accordingly, there is a need for vascular access devices that are easy to implant and access, for delivering medicine and fluids to patients, which are less prone to occlusion and the various limitations outlined above.

### SUMMARY

There is a need for improvements for intraosseous infusion ports and how they are used. The present invention is directed toward further solutions to address this need, in addition to having other desirable characteristics.

In accordance with exemplary embodiments of the present disclosure, an implantable device is provided. The implantable device including: a proximal portion designed for subcutaneous placement, the proximal portion including a concave inlet extending distally toward a first channel; a distal portion designed for intraosseous placement at a site of interest, the distal portion having a second channel terminating in an opening and being co-axially aligned and in fluid communication with the first channel to define a pathway from the proximal portion to the site of interest; and a penetrable seal situated between the first channel and the second channel to minimize backflow from the site of interest into the proximal portion.

In some embodiments, the concave inlet can have a surface defined by a rounded arc which is curved towards a central axis of the pathway. The distal portion can further include one or more retention elements to assist the implantation and anchoring of the distal portion within bone. The proximal portion can further include a recess at a distal end to receive at least one of the penetrable seal and a top end of the intraosseous portion. The proximal portion can further include overhanging ends encasing at least one of the penetrable seal and the top of the distal intraosseous portion. The second channel can include a tapered inlet at a proximal end.

In some embodiments, the penetrable seal can be a funnel shaped with one or more resealable flaps. The one or more resealable flaps can extend distally towards a central axis to direct an insertion device into the second channel.

In accordance with exemplary embodiments of the present disclosure, a system is provided. The system includes, an implantable device having: a proximal portion designed for subcutaneous placement, the proximal portion including a first channel; a distal portion designed for intraosseous placement at a site of interest, the distal portion including a second channel terminating in an opening and which is co-axially aligned and in fluid communication with the first channel to define a pathway from the proximal portion to the site of interest; and a penetrable seal disposed between the first channel and the second channel to minimize backflow from the site of interest into the proximal portion; and a driving mechanism for interfacing with a top the proximal portion for applying axial and rotational force to the implantable device.

In some embodiments, the driving mechanism can include a sheath including a distal opening having an interior shape which is complimentary to an outer shape of the proximal portion to receive the implantable device; and a tube insertable into a lumen of the sheath to adjust a length of the driving mechanism. The tube can be coupled to an insertion mechanism for advancement through a distal end of the sheath. The tube can further include one or more locking posts for traversing within a guide path disposed through the sheath and the one of more locking posts designed for locking in place in the guide path. The driving mechanism can further include a handle having an opening to receive a proximal end of the tube for applying the rotational force. An insertion mechanism can be axially aligned with the first and second channels such that the insertion mechanism can be advanced through the penetrable seal of the device.

In accordance with exemplary embodiments of the present disclosure, a method of placement of a device at a site of interest is disclosed. The method includes providing a device having a proximal portion designed for subcutaneous placement, the proximal portion including a first channel; a distal portion designed for intraosseous placement at a site of interest, the distal portion including a second channel terminating in an opening and which is co-axially aligned and in fluid communication with the first channel to define a pathway from the proximal portion to the site of interest; and a penetrable seal disposed between the first channel and the second channel to minimize backflow from the site of interest into the proximal portion; engaging the proximal portion of the device with an driving mechanism; and applying a rotational force on the driving mechanism to rotationally advance the distal portion of the device into a hardened layer at the site of interest.

In some embodiments, the method can further include inserting an insertion device through the pathway of the device. The proximal portion can include a concave inlet extending distally toward the first channel and the insertion device is directed towards the pathway by the concave inlet. The insertion device can be configured to deliver a substance to the site of interest or collect a substance from the site of interest. After the applying step, the device can be later identified by palpating a center of the device. The driving mechanism can include a distal opening having an interior shape which is complimentary to an outer shape of the proximal portion to receive the device.

In accordance with exemplary embodiments of the present disclosure, an intraosseous port device is provided. The intraosseous port device includes a body having a proximal portion configured and arranged for subcutaneous placement and defining a first channel, and a distal portion designed for intraosseous placement at a site of interest and defining a second channel in fluid communication with the first channel to form a pathway from the proximal portion to the site of interest; a penetrable seal having a dome-shaped portion occluding at least a portion of the first channel; and a retention ring coupled to at least one of the body and the penetrable seal.

In some embodiments, the first channel can include a first portion of a constant first diameter and a second portion with decreasing diameters. The second channel can include a constant second diameter. The constant first diameter can be larger than the constant second diameter. The proximal portion can include a hexagonal lateral cross-section.

In some embodiments, the penetrable seal can include a vertical extension, the vertical extension having an outer diameter that is smaller than the constant first diameter. The dome-shaped portion can have an outer diameter that is equal to the outer diameter of the vertical extension. The dome-shaped portion can have an outer diameter that is smaller than the outer diameter of the vertical extension, and a step is formed between the dome-shaped portion and the vertical extension.

In some embodiments the retention ring can be sized to fit within the first channel. The penetrable seal can be sandwiched between the retention ring and an inner wall of the first channel. The distal portion of the body can include a threaded portion.

In accordance with exemplary embodiments of the present disclosure, a system is provided. The system includes the intraosseous port device; and a driving mechanism including: a port driver having a distal end configured to engage with a proximal portion of the body of the intraosseous port device, and an elongated distal end; a stylet driver receivable within the elongated distal end of the port driver; and a stylet coupled to the stylet driver.

In some embodiments, the stylet driver can be axially translatable relative to the port driver. The stylet can be axially translatable relative to the port driver. The distal end of the port driver can include a socket configured to mate with the proximal portion of the body of the intraosseous port device, the port driver being capable of applying rotational force to the intraosseous port device. The distal end of the port driver can include a shape that compliments the shape of the proximal portion of the intraosseous port device. The distal end of the port driver can include an opening sized to allow the stylet to be advanced therethrough.

In some embodiments, a handle can be configured with an opening to interface with a proximal end of the stylet driver. The driving mechanism can be transitionable between a first condition where the stylet is completely retracted within the port driver, and a second condition where the stylet is at least partially advanced through the port driver. The stylet can extend at least 5 mm from the port driver in the second condition.

In accordance with exemplary embodiments of the present disclosure, an implantable device is provided. The implantable device includes a proximal portion designed for subcutaneous placement and having a first channel defined by a substantially concave shape; a distal portion designed for intraosseous placement at a site of interest and having a second channel in fluid communication with the first channel to define a pathway from the proximal portion to the site of interest; and a penetrable seal situated between the first channel and the second channel to minimize backflow from the site of interest into the proximal portion.

In some embodiments, the concave inlet of the subcutaneous portion can have a rounded arc curved toward a central axis of the subcutaneous portion. The device can further include one or more retention elements to assist the implantation and anchoring of the body section. The subcutaneous portion can further include a recess in a distal end to receive at least one of the penetrable seal and a top end of the intraosseous portion. The subcutaneous portion can include overhanging ends encasing the at least one of the penetrable seal and the top of the distal intraosseous portion.

In some embodiments, an inlet to the second substantially vertical channel can be tapered. The penetrable seal can be a funnel shaped gasket or sock with one or more resealable flaps. The gasket or sock can further include a central axis point aligned with the tips of the one or more resealable flaps to direct an insertion device into the second substantially vertical channel. The subcutaneous portion can further include at least one cavity creating a space between a top of the intraosseous portion and a body of the subcutaneous portion.

In accordance with exemplary embodiments of the present disclosure, a system is provided. The system includes an implantable device including: a proximal portion designed for subcutaneous placement and having a first channel defined by a substantially concave shape; a distal portion designed for intraosseous placement at a site of interest and having a second channel in fluid communication with the first channel to define a pathway from the proximal portion to the site of interest; and a penetrable seal situated between the first channel and the second channel to minimize backflow from the site of interest into the proximal portion; and a driving mechanism for interfacing with a top of the subcutaneous portion for applying rotational force to the device.

In some embodiments, the driving mechanism can include a sheath having an opening complimenting a shape of top of the subcutaneous portion for interfacing with the device; and a tube insertable into a lumen of the sheath to control a length of the driving mechanism. The tube can be coupled to an insertion mechanism for advancement through a distal end of the sheath. The tube can further include one or more locking posts for traversing within a guide path within the sheath and locking in place in one or more locking slots in the sheath.

In some embodiments, the driving mechanism can further include a handle configured with an opening to interface with a proximal end of the tube for applying the rotational force. The handle can further include a button for decoupling the handle from the proximal end of the tube. The opening in the handle can interface with a distal end of the device.

In some embodiments, an insertion mechanism can be advanced through the penetrable seal of the device when the distal end of the device is interfaced with the handle. The concave inlet of the subcutaneous portion can have a rounded arc curved toward a central axis of the subcutaneous portion. The penetrable seal can be a funnel shaped gasket or sock with one or more resealable flaps and a central axis point aligned with the tips of the one or more resealable flaps to direct an insertion device into the second substantially vertical channel.

### BRIEF DESCRIPTION OF THE FIGURES

These and other characteristics of the present invention will be more fully understood by reference to the following detailed description in conjunction with the attached drawings, in which:

FIG. 1A is a cross-section side view of an implantable intraosseous infusion port (TOP) device in accordance with an embodiment of the present disclosure;

FIG. 1B is a cross-section side view of an implantable intraosseous infusion port (TOP) device in accordance with an embodiment of the present disclosure;

FIG. 1C is a cross-section side view of an implantable intraosseous infusion port (TOP) device in accordance with an embodiment of the present disclosure;

FIG. 1D is a cross-section side view of an implantable intraosseous infusion port (TOP) device in accordance with an embodiment of the present disclosure;

FIG. 2A is a top view of a penetrable seal for use in an implantable intraosseous infusion port (TOP) device in accordance with an embodiment of the present disclosure;

FIG. 2B is a top view of a penetrable seal for use in an implantable intraosseous infusion port (TOP) device in accordance with an embodiment of the present disclosure;

FIG. 2C is an isometric view of a penetrable seal for use in an implantable intraosseous infusion port (TOP) device in accordance with an embodiment of the present disclosure;

FIG. 2D is an isometric view of a penetrable seal for use in an implantable intraosseous infusion port (TOP) device in accordance with an embodiment of the present disclosure;

FIG. 2E is an isometric view of a penetrable seal for use in an implantable intraosseous infusion port (TOP) device in accordance with an embodiment of the present disclosure;

FIG. 2F is an isometric view of a penetrable seal for use in an implantable intraosseous infusion port (TOP) device in accordance with an embodiment of the present disclosure;

FIG. 3 is an example cross-section side view of an implantable intraosseous infusion port (TOP) device in accordance with the present disclosure;

FIG. 4A-4I illustrate various examples of an implantable intraosseous infusion port (TOP) devices in accordance with the present disclosure;

FIG. 5 is an example isometric view of a driving mechanism in accordance with the present disclosure;

FIGS. 6A and 6B are example isometric views of a drive shaft for a driving mechanism in accordance with the present disclosure;

FIGS. 6C, 6D, 6E, and 6F are example cross-sectional side views of a drive shaft for a driving mechanism in accordance with the present disclosure;

FIG. 7A is an example cross-sectional side view of a handle for a driving mechanism in accordance with the present disclosure;

FIGS. 7B and 7C are example isometric views of an assembly process for a driving mechanism in accordance with the present disclosure;

FIGS. 8A, 8B and 8C are example isometric views of an assembly process for a driving mechanism in accordance with the present disclosure;

FIGS. 9A-9L are schematic cross-sectional views showing the implantation of the IOP device in accordance with one embodiment of the present disclosure;

FIGS. 10A-10H are schematic perspective views of one example of a driving mechanism in retracted and advanced conditions, respectively; and

FIGS. 11A-11I are schematic side views showing the use of an assembly to deliver and fix a port according to the present disclosure.

### DETAILED DESCRIPTION

An illustrative embodiment of the present invention relates to implantable intraosseous infusion port (TOP) devices and method of use to deliver fluids and medicines to bone marrows. As used herein, the terms “TOP device,” “infusion port,” and “port” may be used interchangeably. The various embodiments of the present disclosure can be used to provide short- or long-term access to bone marrow cavities. The TOP device can be designed for placement within tissue and into bone. The placement can be percutaneous or subcutaneous within the tissue while providing visual and tactile feedback as to the position of the device relative to the bone. In some embodiments, the top of the device can be concave in design to assist in guiding a needle into a central channel of the device, even when the device is not directly visible under tissue. The device can also include a penetrable seal positioned at the base of the concave inlet to protect fluid, or other materials, from inadvertently leaving or entering the channel. The distal end of the channel can include retention elements to secure the device within the tissue and/or bone.

In some embodiments, the device can be placed into tissue and bone by using an insertion device. In some embodiments, the drive shaft can include an opening for securing the device thereto for positioning and placement into a desired location. The insertion device can be a multi component system including a drive shaft and handle. The handle can couple to the drive shaft to provide a point in which an application of force can be applied by the user and translated to the drive shaft and then to the device. For example, by turning the handle, a rotational force can be applied to the device via the drive shaft to cause the device to rotate into tissue/bone. The drive shaft can include a sheath and a tube to provide an adjustable length for different applications and for use at different locations. In some embodiments, the tube can also include an insertion mechanism to assist in the placement of the device into tissue/bone.

Various example embodiments will be described more fully hereinafter with reference to the accompanying drawings, in which some example embodiments are shown. The present inventive concept may, however, be embodied in many different forms and should not be construed as limited to the example embodiments set forth herein. Rather, these example embodiments are provided so that this disclosure will be thorough and complete, and will fully convey the scope of the present inventive concept to those skilled in the art. In the drawings, the sizes and relative sizes of layers and regions may be exaggerated for clarity. Like numerals refer to like elements throughout.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

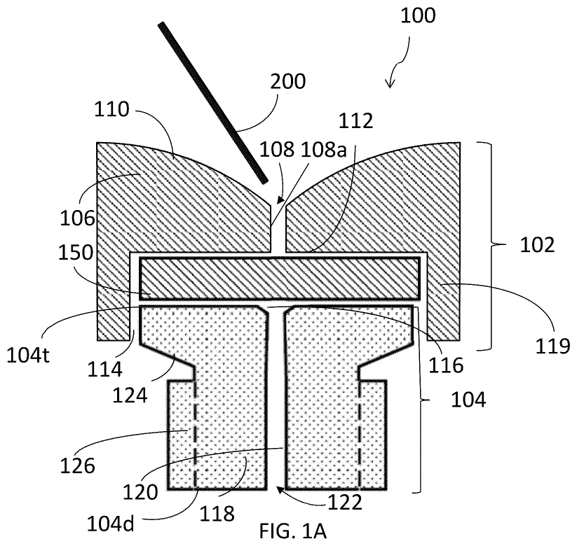

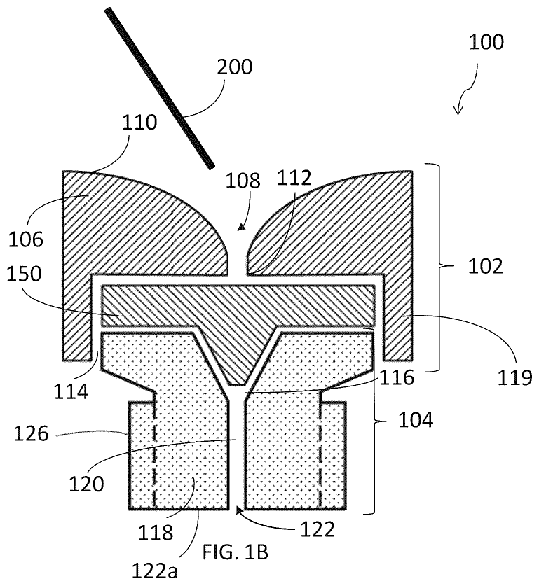

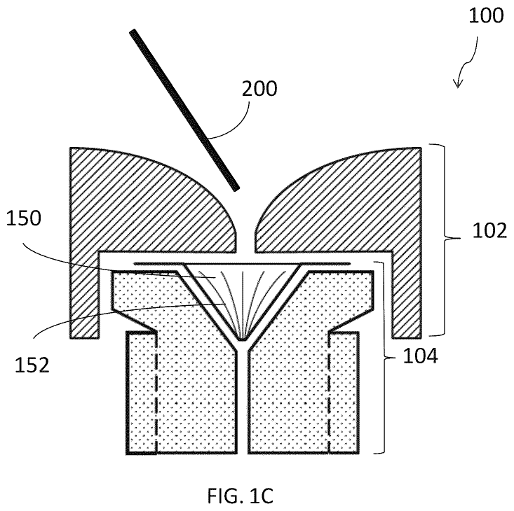

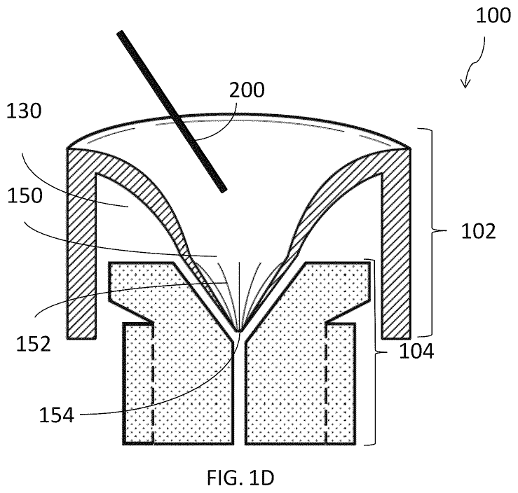

FIGS. 1A through 151, wherein like parts are designated by like reference numerals throughout, illustrate an example embodiment or embodiments of improved operation for implantable intraosseous infusion port (TOP) devices, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

Referring to FIGS. 1A-1D, cross-sectional views of example IOP devices 100 are depicted in accordance with various embodiments of the present disclosure. Referring to FIG. 1A, in some embodiments, the IOP device 100 can generally include a proximal portion 102, which, in an embodiment, can be placed subcutaneously, a distal portion 104, which, in an embodiment, can be positioned intraosseously, and a seal 150 disposed therebetween. The proximal portion 102 may be designed to reside in soft tissues and can be accessible through the skin while the distal portion 104 can be designed to pass through, and can be anchored to, the cortex of a bone, with its distal end 104d residing within, or at least flush with, the marrow cavity. The combination of the proximal portion 102 and the distal portion 104 can provide an improved mechanism for accurately introducing an insertion device 200 into tissue. For example, the combination of the subcutaneous proximal portion 102 and the intraosseous distal portion 104 can provide a mechanism in which an insertion device 200, in the form of a needle, can be guided into a desired site, such as tissue or bone marrow, at an optimal angle defined by the proximal portion 102 and the distal portion 104. As discussed herein, the terms vertical and horizontal make reference to the vertical and horizontal orientation of elements of the device 100 as depicted in FIGS. 1A-1D and are not intended to be limited to the usage of the device 100 to any particular orientation.

In some embodiments, the proximal portion 102 can include a body portion 106 with channel 108 extending therethrough from an inlet 110 to an outlet 112. The body portion 106 can effectively form a wall around the channel 108 in a vertical direction. In this fashion, the channel 108 may be a substantially straight pathway disposed within the wall, where the outlet 112 can be an opening at the distal end of the channel 108 at a plane that is flush with the distal end of the wall. The channel 108 can provide a continuous pathway from the proximal portion 102 into which an insertion device 200 can be inserted through the proximal portion 102. It should be appreciated that the surface of the inlet of body portion 106 can have any geometric shape so long as the shape can facilitate an easy insertion of an insertion device 200. In some embodiments, the inlet end of the channel 108 can have a diameter at one end that is measurably greater than the diameter at an opposing end to create an inlet 110 that reduces in size from one end to the other. For example, the channel 108 can be sized and dimensioned to extend from inlet 110 and the outlet 112 to create a substantially funnel shape. The inlet 110 may provide access to the channel 108 and can be accessed through the skin after the implantation of the IOP device 100.

In some embodiments, the channel 108 can include a continuous slope directed toward the outlet 112 such that an insertion device 200 contacting the body portion 106 would slide along the slope and be directed toward the outlet 112. As depicted in FIGS. 1A-1D, in some embodiments, the channel 108 can be substantially convex in shape. The slope can include any combination of shapes including convex or concave shapes. In some embodiments, the channel 108 can include a substantially vertical portion 108a, which can be linear, located near and extending to the outlet 112. The substantially vertical portion 108a can be sized and dimensioned to correctly orient, point, or direct an insertion device 200 being directed by the slope of the channel 108 into the outlet 112 at a substantially vertical angle into and through the distal portion 104. The channel 108 may also be of any shape or size so long as fluids and/or medicines can be delivered through the proximal portion 102. In some embodiments, the channel 108 may be a channel passing through the body portion 106 of the IOP device 100, or the channel 108 may be in the form of a fluid reservoir similar to that of a traditional implantable vascular access port.

In some embodiments, the proximal portion 102 can be sized and shaped to fit over and surround at least a top portion 104t of the distal portion 104. For example, the proximal portion 102 can have a recess 114 in its distal end to receive at least one of a penetrable seal 150 and a top end of the distal portion 104. In the illustrated embodiment, the recess 114 encloses both the penetrable seal 150 and the top portion 104t of the distal portion, with overhanging ends 119 encasing the penetrable seal 150 and the top portion 104t of the distal portion 104, as depicted in FIGS. 1A and 1B. In some embodiments, the recess 114 can be sized and shaped to conform to the shape(s) of the penetrable seal 150 and the top of the distal portion 104. The size of the recess 114 can be provided to provide a tight friction fit or a loose fit with at least one of the penetrable seal 150 and the top of the distal portion 104. In some embodiments, the recess 114 can be sized to provide room for vertical displacement of the penetrable seal 150 upon introduction of an insertion device 200 into the penetrable seal 150. In some embodiments, the proximal portion 102 can be constructed from rigid or semi-rigid materials, such as metal, plastic or any other such biocompatible material. When the TOP device 100 is fully assembled and implanted, the outlet 112 of the channel 108 can be axially aligned with, and be in fluid communication with, an inlet 116 of the distal portion 104 of the device 100.

Continuing with FIGS. 1A-1D, in some embodiments, the distal portion 104 of the device 100 may include a body section 118 and a channel 120, the channel 120 extending between an inlet 116 and an outlet 122. The channel 108 from the proximal portion 102 and the channel 120 from the distal portion 104 can be designed to be in substantial co-axial alignment to define a pathway for a surgical instrument to be directed from the proximal portion 102 through the distal portion 104 and to the site of interest. In some embodiments the channel 108 from the proximal portion 102 and the channel 120 from the distal portion 104 can be substantially the same diameter.

In some embodiments, while the channel 120 can travel in a substantially vertical direction extending from the inlet 116 to the outlet 122. For example, the channel 120 can create a substantially straight pathway extending from the outlet 112 of the proximal portion 102 out the distal end of the distal portion 104. In some embodiments, as depicted in FIGS. 1A-1D, the inlet 116 can have a tapered entry point. The tapered entry point of the inlet 116 can be designed to assist in ensuring that an insertion device 200 is properly directed into the channel 120.

In some embodiments, the channel 120 can be an open-ended channel terminating at the outlet 122. The outlet 122 may reside at a distal end of the body section 118, oriented along a same direction as the channel 120. Furthermore, the outlet 122 can be an opening on a plane 122a that lies flush with the distal ends of the body section 118, where the outlet 122 may be oriented in the same direction as the channel 120. In addition, the inlet 116 of channel 120 may be coupled to the outlet 112 of the channel 108, where the channel 120 and the channel 108 effectively create a substantially straight pathway where an insertion device 200 (e.g., needle) can travel through the pathway to reach the bone marrow cavity.

In some embodiments, the distal portion 104 can effectively function as an anchor portion for the IOP device 100. For example, the distal portion 104 can be sized and shaped to anchor the device 100 into a bone to provide access to a bone marrow cavity. The distal portion 104 can have a proximal retaining lip 124 that has a larger diameter than the distal end to create the overall shape of the body section 118. For example, the proximal retaining lip 124 of the body section 118 can be similar in shape to a substantially truncated cone or polygonal shape mated with a substantially cylindrical shape of the distal end of the of the body section 118, similar to the cross-sectional view of FIGS. 1A and 1B.

In some embodiments, the distal portion 104 may possess a proximal retaining lip 124 or other similar features for limiting the distal intraosseous portion's 104 depth of penetration into the bone marrow. The retaining lip 124 can include any combination of shapes. For example, the lip 124 can be a sloped surface (as depicted in FIG. 1A) extending radially inward from a proximal end towards a distal end, a substantially flat surface extending perpendicularly in relation to the channel 120, a rounded surface, or any combination thereof. In some embodiments, the distal portion 104 may be constructed of any rigid material that can pass through and be anchored to the bone, and it can be of any length that is capable of traversing the cortical bone. In some embodiments, the proximal portion 102 and the distal portion 104 can be discrete components and implanted into a patient separately. In some embodiments, the proximal portion 102 and the distal portion 104 may be discrete components connected together using means commonly practiced in the art (i.e., glue, thermal bonding, welding, melting, friction fit, etc.) and implanted together into a bone. In some embodiments, the proximal portion 102 and the distal portion 104 may be parts to a single structure and may be implanted as a single device into a bone.

In some embodiments, the distal portion of the IOP device 100 can include retention elements 126 to be positioned substantially adjacent with at least a portion of the body section 118 of the distal portion 104. The retention elements 126 can be provided to assist the implantation and anchoring of the body section 118 within a bone, tissue, or other material. In some embodiments, the retention elements 126 can extend radially outward from an outer surface of the distal portion 104. For example, the retention elements 126 can be radially extending ridges, threads, or other protrusions.

The components of the IOP device 100 can be sized and scaled for any combination of purposes. For example, as a non-limiting example, the proximal portion 102 may have a diameter range from about 8 mm to about 20 mm and the distal portion 104 may have a diameter range from about 5 mm to about 15 mm. The height of the proximal portion 102 and height of the distal portion 104 can vary depending on the specific application the IOP device 100 will be used for. The inlet 110 may have an upper diameter range from about 5 mm to about 15 mm and the outlet 112 may have a lower diameter range from about 3 mm to about 8 mm and a slope range from about 30 to about 60 degrees. In some embodiments, the channel 120 may range from about 1 mm to about 5 mm in diameter, wide enough for passing through an insertion device 200, e.g., a needle, designed for medical applications.

In some embodiments, the IOP device 100 can include a penetrable seal 150 housed between the proximal portion 102 and the distal portion 104. The seal 150 can provide a penetrable layer through which an insertion device 200 can penetrate while limiting any fluid or materials from transitioning from the proximal end to the distal end of the device 100. For example, the seal 150 may be penetrated by an insertion device 200 such as a needle to reach a bone marrow cavity. The seal 150 may be a rubber septum, a membrane, or any material that can be penetrated while maintaining a seal separating channel 108 and the channel 120. For example, the seal 150 can be designed to have a diameter large enough to seal off the channel 108 such that a reflux of the infused fluid into the subcutaneous tissue (e.g., through channel 120) may be prevented. All, or a portion, of the inlet 116 may be covered with the seal 150, through which an insertion device 200 may pass to access the channel 120, to prevent reflux of the infused fluid into the subcutaneous tissue. Further, in some embodiments, the proximal portion 102 can include a retaining ring to hold the seal 150 in place.

Referring to FIGS. 1A and 1B, in some embodiments, the seal 150 can be a penetrable, and self-healing, septum positioned between the proximal portion 102 and the distal portion 104 to minimize, or prevent, backflow from the site of interest into the proximal portion. The seal 150 can be any combination of penetrable material, such as rubber, silicone, elastomer, etc. shaped to fit between the proximal portion 102 and the distal portion 104 of the device 100. In some embodiments, the seal 150 can be sized and shaped to fit within the recess 114 of the proximal portion 102 and can rest on a top surface of the distal portion. The recess 114 and the seal 150 can be a sufficient size to allow lateral, and/or axial, displacement of the seal 150 within the recess 114 during insertion of a device therein. For example, when a needle is inserted through the seal 150, there is a sufficient size differential between the seal 150 and the recess 114 to allow the seal 150 to displace within the recess 114 while the needle is inserted therein.

Referring to FIG. 1A, in some embodiments, the seal 150 can be a septum that is substantially cylindrical in shape, akin to a disk, to be situated within the recess 114 and substantially adjacent to the top portion 104t of the body section 118. The seal 150 may be secured between and/or within either of the proximal portion 102 or distal portion 104 using any combination of mechanisms. For example, the seal 150 can be held in place via a friction fit, pressure fit, adhesive, mechanical fastener, weld, etc., or a combination thereof.

Referring to FIG. 1B, in some embodiments, the inlet 116 of the channel 120 can have a larger diameter than the remainder of the channel 120 and the outlet 122. The larger diameter inlet 116 can be sloped toward the channel 120 to create a substantially funnel shape with straight sides. In some embodiments, as seen in FIG. 1B, the seal 150 can be shaped to conform to the shape of the recess 114 of the proximal portion 102 and the exaggerated inlet 116 of the distal portion 104. For example, when the recess 114 is substantially cylindrical shaped and the inlet 116 is substantially funnel shape, the seal 150 can include a cylindrical shape extending into a substantially truncated cone shape.

Referring to FIG. 1C, in some embodiments, the seal 150 can be penetrable cone sandwiched between the proximal portion 102 and the distal portion 104. The seal as a penetrable cone can have a scored design with or without a septum or membrane. The penetrable cone can include any combination of shapes and designs that allows penetration by an insertion device 200 while also preventing flow back from the insertion site (e.g., bone marrow) back into the channel 108. The penetrable cone can also be constructed from any combination of materials, for example, a metallic sealing compound, plastics, silicones, etc. In some embodiments, the seal 150 can include one or more resealable flaps 152 that can be moved out the path of the insertion device 200 when the insertion device contacts the flaps 152. In some embodiments, the one or more flaps 152 can be created by slits formed in the distal conical portion 156. The one or more flaps, in some embodiments, can be aligned along a central axis point 154 in which the tips of the flaps meet. The central axis point 154 can be substantially aligned with at least one of the channel 108 outlet 112 and the channel 120 inlet 116.

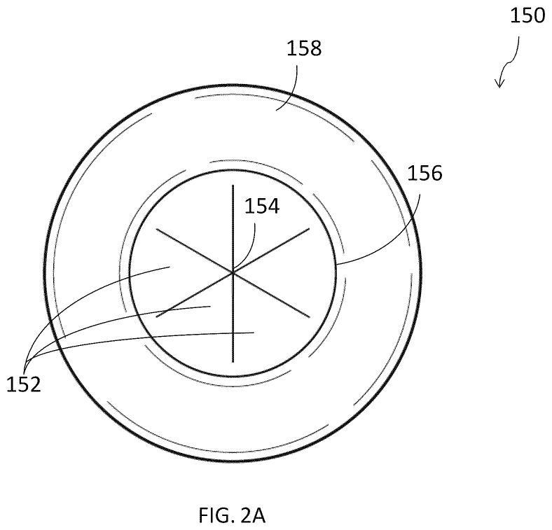

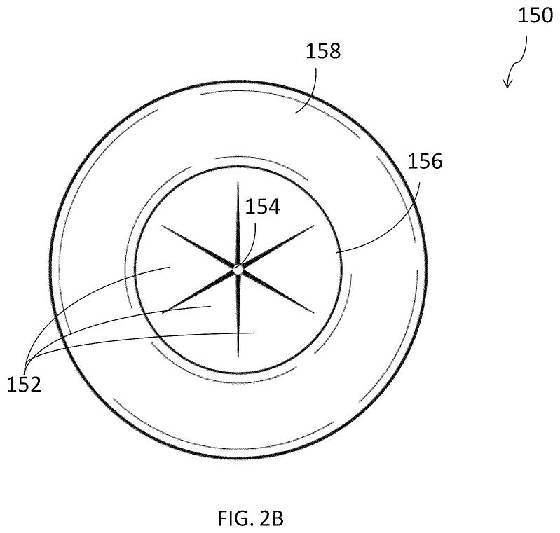

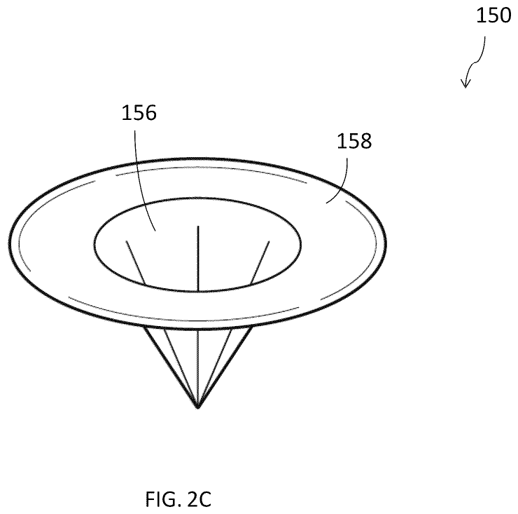

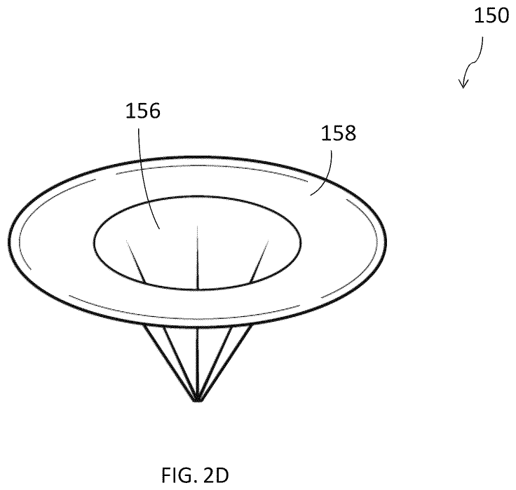

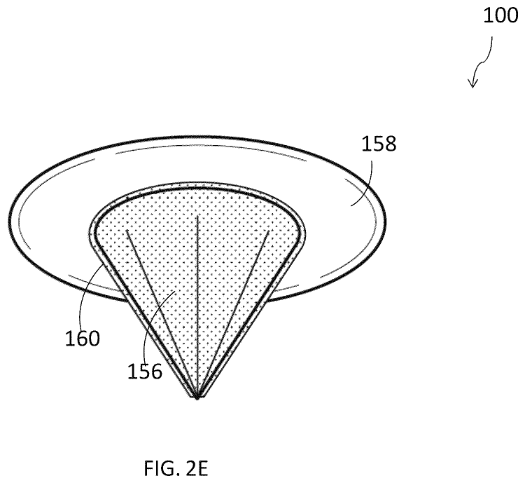

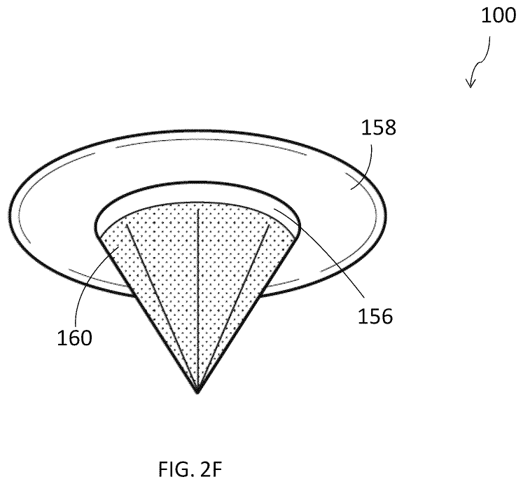

Referring to FIGS. 2A and 2B, top views of example seals 150, for example, the seal 150 provided in FIG. 1C, are depicted. As depicted in FIGS. 2A and 2B, the seal 150 can be substantially upturned round cone shaped and can include a plurality of flaps 152 to allow an insertion device 200 to be inserted therethrough. Although FIGS. 2A and 2B show six flaps, any combination of flaps 152 can be used. The flaps 152 can be sized, shaped, and made from a material to move out of the way when pressure is applied by an insertion device 200 then reform back to the original shape (cone) when the insertion device 200 is removed. Similarly, the flaps 152 can be designed to move aside while also conforming to the shape of the insertion device 200 to substantially maintain a seal and division between the channel 108 and channel 120. Referring to FIG. 2B, in some embodiments, the seal 150 can have an aperture at the central axis point 154. The aperture at the central axis point 154 may aid in directing the insertion device 200 through a desired location at the center of the seal 150, to ensure that the insertion device 200 is directed to the channel 120 of the distal portion 104.

Referring to FIGS. 2C and 2D, in some embodiments, the seal 150 can include a conical distal portion 156 and a substantially flat, disk shaped, proximal portion 158. The substantially flat proximal portion 158 can extend from the base of the cone shape of the distal portion 156. The substantially proximal portion 158 can follow the contour of the base of the distal portion 156 to create a circular, or disk, shape. Although the proximal portion 158 and distal portion 156 are discussed as being conical and circular in shape, any combination of shapes could be used without departing from the scope of the present invention. For example, the distal portion 156 can be a pyramid shape and the proximal portion 158 can be a rectangular shape extending therefrom. Such non-circular shaped seals 150 In some embodiments, the overall combined seal 150 shape can be designed to fit within the shapes created by the proximal portion 102 and the distal subcutaneous portion 104. For example, the seal 150, as depicted in FIGS. 2A-2F, can be sized, and shaped to fit within the recess 114 of the proximal portion 102 and the sloped inlet 116 of the channel 120 within the distal subcutaneous portion 104, as depicted in FIG. 1C. Referring to FIGS. 2C and 2D, top isometric views of an example seal 150 are depicted.

Referring to FIGS. 2E and 2F, bottom isometric views of an example seal 150 are depicted. In some embodiments, as seen in FIG. 2E, the distal portion 156 can be at least partially perforated. In other words, the distal portion 156 can include a series of perforations, or through holes. A perforated distal portion 156 can enable controlled fluid transfer from the channel 108 to distribute through to the channel 120, in conjunction with or in place of an insertion device 200. Referring to FIG. 2F, in some embodiments, the distal portion 156 can have a permeable layer 160 situated around the conical portion. The permeable layer 160 can be designed to prevent flow back through the device 100 from the bone marrow cavity, for example. The permeable layer 160 can include any combination of materials and can be coupled to the distal portion 156 of the seal 150 using any combination of techniques. For example, the permeable layer 160 can be an elastomer sock positioned over the distal portion 156 via a friction fit. Referring to FIG. 2F, in some embodiments, the permeable layer 160 can be a coating applied to the distal portion 156 of the seal 150. The coating can be similar in material with respect to FIG. 2E. Regardless of design, the seal 150 can be designed from any combination of materials, for example, elastic biocompatible materials.

Referring back to FIG. 1D, in some embodiments, the seal 150 can be an integrated portion of the proximal portion 102. The distal portion 104 is substantially the same as the distal portion of FIG. 1C, however the embodiment of FIG. 1D provides for an integrated proximal portion with a seal. Thus, only the proximal portion will be discussed for the sake of brevity. When the proximal portion 102 and the seal 150 are integrated, the size and shape of the proximal portion 102 can be similar than that of the proximal portion 102 when it is not integrated with the seal 150, for example, as discussed with respect to FIGS. 1A and 1B. The integrated seal 150 can be designed as a penetrable cone which can include any combination of shapes and designs that allows penetration by an insertion device 200 while also preventing flow back from the insertion site (e.g., bone marrow) back into the channel 108. In some embodiments, the seal 150 can include one or more resealable flaps 152 that can be moved out the path of the insertion device 200 when the insertion device contacts the flaps 152. The one or more flaps, in some embodiments, can be aligned along a central axis point 154 in which the tips of the flaps meet. The central axis point 154 can be substantially aligned with at least one of the channel 108 outlet 112 and the channel 120 inlet 116.

Continuing with FIG. 1D, in some embodiments, when the proximal portion 102 and the seal 150 are integrated, it can include at least one cavity 130 creating a space between a top of the distal portion 104 and the body of the proximal portion 102.

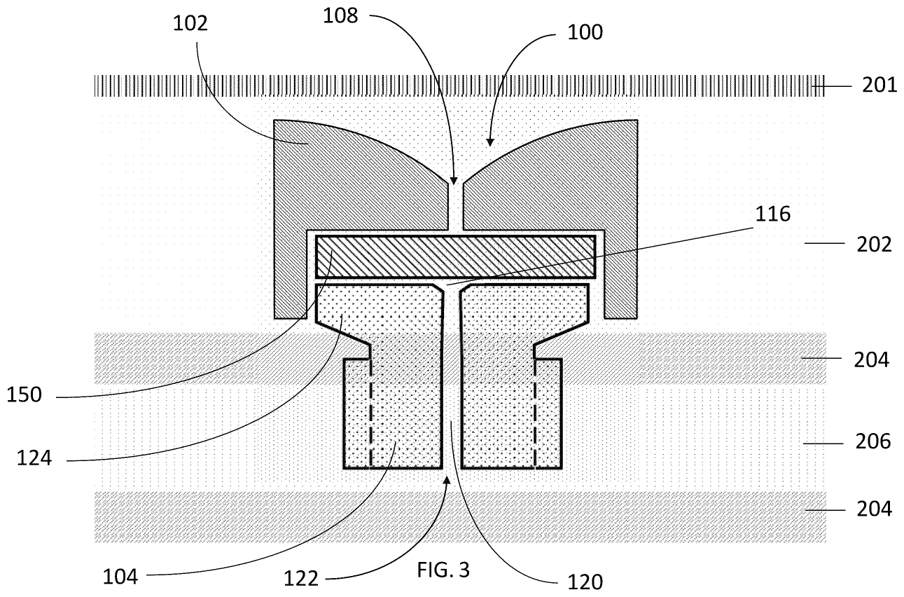

Referring to FIG. 3, an example cross-sectional illustration of the device 100, of FIG. 1A, implanted within subcutaneously within tissue 202 and anchored into a cortex layer 204 of a bone is depicted. When fully implanted subcutaneously into tissue 202, as illustrated in FIG. 3, the proximal portion 102 of the IOP device 100 can be substantially submerged underneath a layer of skin 201 and within a layer of subcutaneous tissue 202. In some embodiments, the proximal portion 102 may be entirely submerged within tissue 202 and may be coupled to the distal portion 104 at the retaining lip 124 where the channel 108 can be aligned with the channel 120. The channel 108, in combination with the channel 120, can be configured to provide a continuous pathway that allows an insertion needle or catheter to extend towards the bone marrow cavity 206, when the IOP device 100 is implanted into the cortex layer 204 of the bone. In some embodiments, the distal portion 104 may be anchored through a cortex layer 204 and into the bone marrow 206, such that the outlet 122 may be in direct contact with the bone marrow 206. An insertion device 200, such as a needle, may be used to penetrate through the skin 201, through channel 108 and into the septum 150 covering the inlet 116 into the channel 120 and deliver and/or extract fluids within the bone marrow through the outlet 122.

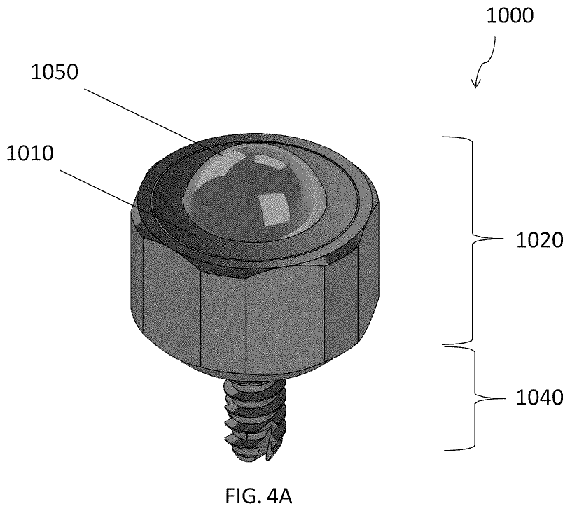

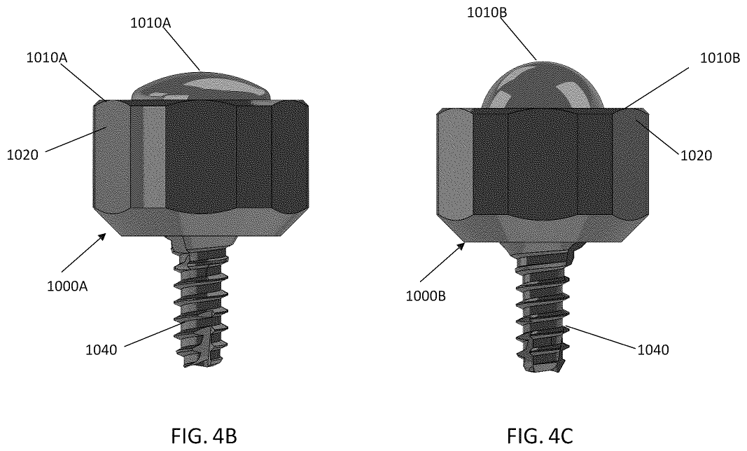

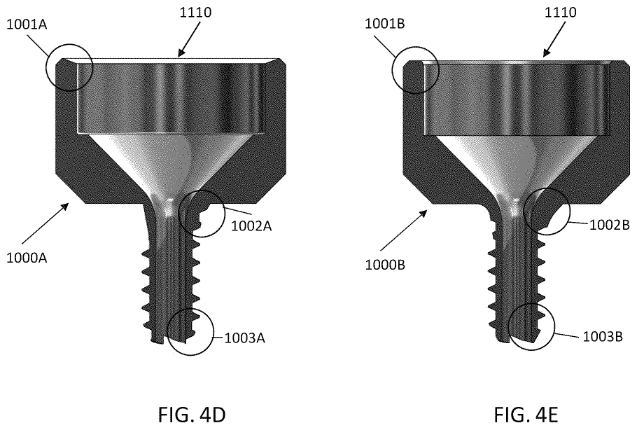

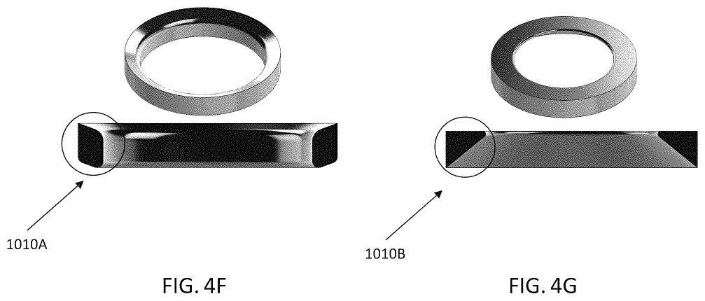

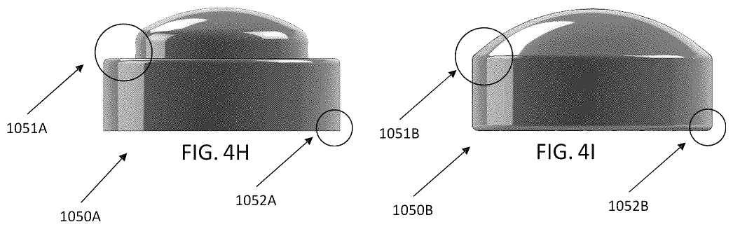

Referring to FIG. 4A, an example isometric view of one alternative example of an IOP device 1000 is depicted. The proximal portion 1020 and the distal portion 1040 can include any combination of shapes. Although the proximal portion 1020 of the device 1000 is illustrated in FIG. 4A as having a hexagonal shape, it should be appreciated that proximal portion 1020 can be provided with any geometric shape. The embodiment of FIG. 4A can be substantially the same as the embodiment of FIG. 1A, with the exception of the seal 1050 and the inclusion of a retention ring 1010. In place of a disk shaped seal 150, a spherical, or semispherical, seal 1050 can be housed within the proximal portion 1020, such that a portion of the spherical seal 1050 extends proximally above the proximal portion 1020. For example, the portion of the seal 1050 extending proximally can provide for a tactile indication of the IOP device 1000 under the skin 201.

Variations of IOP device 100 are possible, similar to the IOP device 100 of FIG. 4A. For example, FIGS. 4B and 4C illustrate two exemplary port assemblies shown side-by-side. Each of ports 1000A and 1000B generally include a proximal portion 1020, a distal portion 1040, a retention ring 1010A, 1010B, and a septum 1050A, 1050B. The various components may include any of the materials described above. As compared to the IOP device of FIGS. 1A-1D, the port assemblies 1000A and 1000B can include the additional retention ring 1010A, 1010B to retain the differently shaped septum 1050A, 1050B within the proximal portion 1020.

One distinction between the two exemplary ports 1000A and 1000B, as best shown in FIGS. 4D and 4E, can include a decreased chamfer size and angle of the inlet 1110 at the top location 1001B, a collar which can be formed from an increase in material adjacent the stem-to-body connection to add strength at location 1002B, and a modified chamfer at location 1003B to maintain adequate wall thickness and uniform minor diameter of threads, when compared to locations 1001A, 1002A, 1003A of port 1000A, respectively. As shown in FIGS. 4F and 4G, ports 1000A and 1000B may include retention rings 1010A or 1010B, respectively. Retention ring 1010B may include a decreased inner diameter to improve septum retention and/or modified cross section geometry to improve septum retention. The retention ring 1010A, 1010B can be retained within the respective ports 1000A, 1000B by means of an adhesive, welding, thermal bonding, interference fit, or other chemical or mechanical means. The retention ring can be sized and shaped to retain the seals 1050A, 1050B within the respective ports 1000A, 1000B to ensure that the ports 1000A, 1000B do not allow for a backflow of fluid, or other materials, through the ports 1000A, 1000B from a bone marrow cavity.

Finally, variations of the septa (i.e., more than one septum) are possible. FIGS. 4H and 4I illustrate two septa 1050A and 1050B. Septum 1050A includes a vertical extension and a dome-shaped member that form a step therebetween, while septum 1050B includes a dome-shaped member that includes vertical extensions from its outer diameter. Compared to septum 1050A, septum 1050B includes a modified geometry to remove stress concentration when under pressure and improve retention at location 1051B and added fillet at bottom at location 1052B to facilitate installation during assembly when compared to locations 1051A and 1052A.

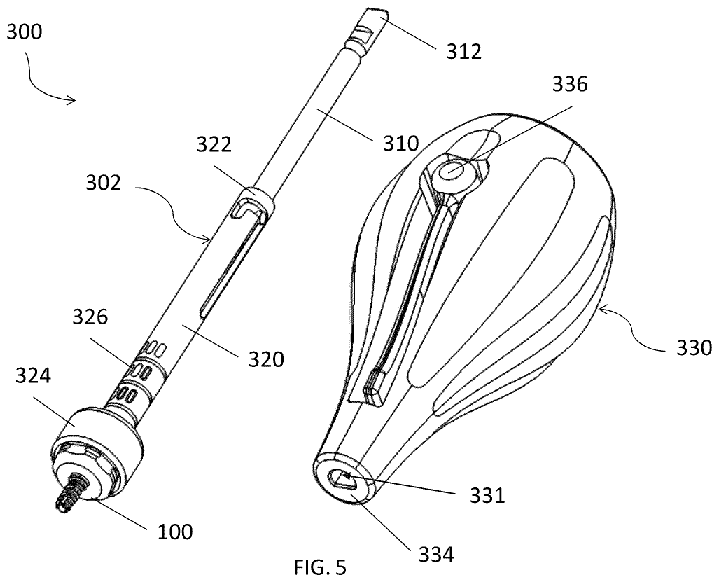

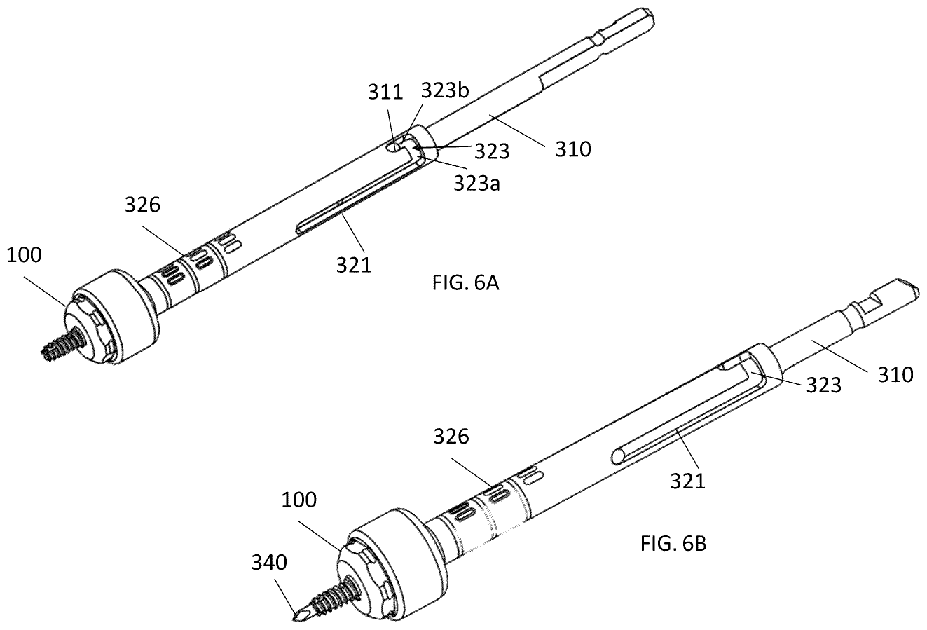

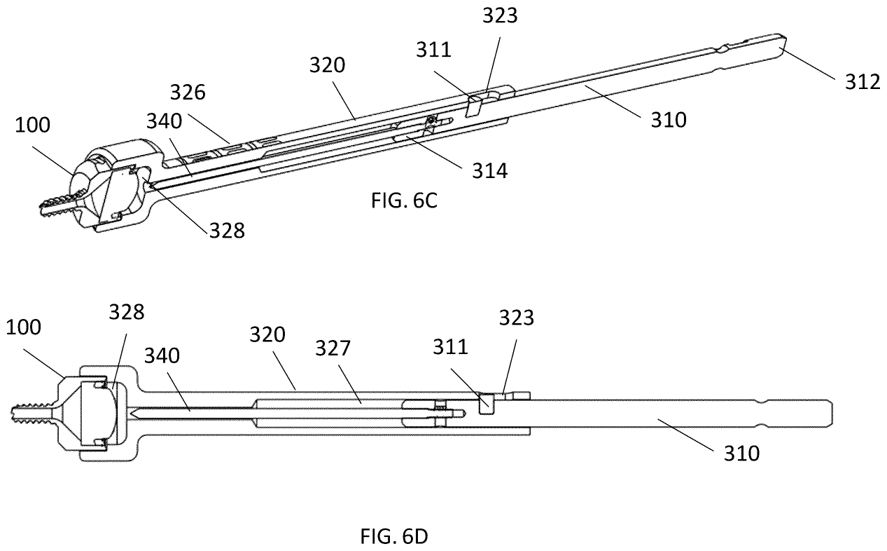

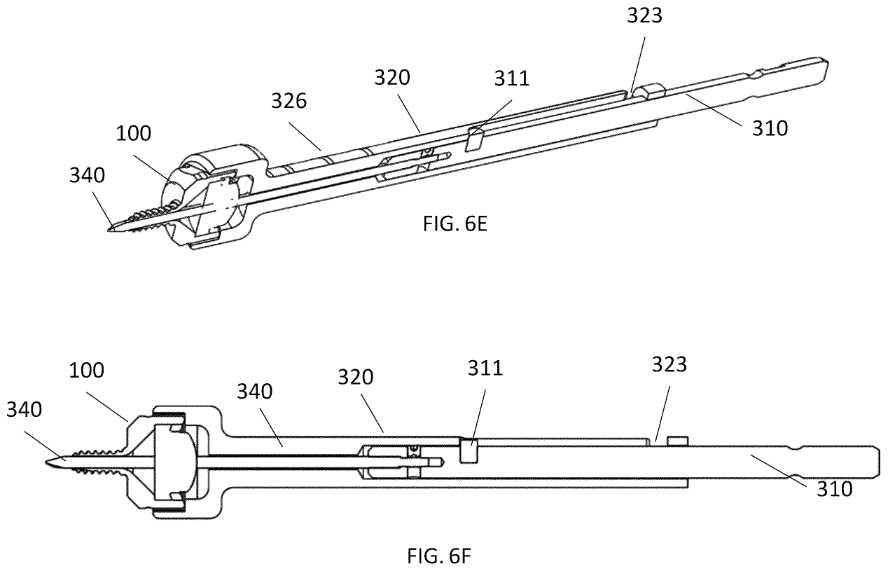

Referring to FIGS. 5-6F, in some embodiments, the device 100 can be implanted into tissue and/or bone using a driving mechanism 300. In some embodiments, the driving mechanism 300 can include two subassemblies, a drive shaft 302 and a handle 330. The combination of the drive shaft 302 and the handle 330 can be designed for advancing the IOP device 100 into a desired location in the bone. For example, the IOP device 100 can be designed to interface with a distal end 324 of the driving mechanism 300 and an application of an axial and rotational force can be applied to the device 100 via the driving mechanism 300. The driving mechanism 300 can be used to place the device 100 at the desired location outside or within the body, for example, for percutaneous, subcutaneous, etc. placement.

The driving mechanism 300, in some embodiments, may include a drive shaft 302 having an inner tube 310 and an outer sheath 320. The inner tube 310 can be designed to be situated within the outer sheath 320. In some embodiments, the tube 310 can be sufficiently rigid to effectively act as a drill bit or needle to penetrate through overlying tissue and into bone. In some embodiments, the sheath 320 can be substantially cylindrical and rigid, and can include a proximal end 322 and a distal end 324. In some embodiments, the sheath 320 can include an open-ended channel or lumen 327 centrally located and extending from the proximal end 322 to the distal end 324. At least a portion of the lumen 327 can be designed to correspond to the outer profile of the tube 310. In some embodiments, the tube 310 can be slidably nested within sheath 320 and be extendable from a proximal end 322 of sheath 320.

As configured, the overall length of the driving mechanism 300 can be adjusted to accommodate different overlying tissue thicknesses (e.g., by adjusting a position of the tube 310 within the sheath 320). The lumen 327 of the sheath 320 can include any combination of sizes and shapes known in the art receive the tube 310 therewithin and enable the tube 310 to telescopically slide and rotate within the lumen of the sheath 320.

In some embodiments, a distal end 324 of the sheath 320 can be sized and shaped to receive and hold the proximal portion 102 of the TOP device 100. The distal end 324 of the sheath 320 can include any combination of shapes that include a recess which compliments the shape of the proximal portion 102 of the IOP device 100. The distal end 324 of the sheath 320 can be configured to provide a surface for interacting with a device (e.g., device 100) to apply torque to the device via driving mechanism 300. For example, if the proximal portion 102 of the TOP device 100 is hexagonal, then the recess 328 within the distal end 324 of the sheath 320 can be hexagonal.

In some cases, the shape of the recess 328 and the proximal portion 102 may not be complimentary shapes and can include any combination of shapes that will enable the sheath 320 to grip, hold, and/or adjust the TOP device 100. With the recess 328 of the distal end 324 of the sheath 320 matching the proximal portion 102 of the TOP device 100, the sheath 320 can be used to hold, move, and apply force to the TOP device 100. For example, the sheath 320 can be used to position the TOP device 100 at a desired location then apply a rotational force to assist in the insertion the TOP device 100 at that location. In some embodiments, the sheath 320 can include a one or more visual indicators 326 thereon. The one or more visual indicators 326 can be used to provide visual feedback to a user for various aspects of the drive shaft 302. For example, the one or more indicators 326 can have numerous lines with numerical indicators for a depth of penetration by the drive shaft 302.

In some embodiments, the tube 310 can be substantially cylindrical and rigid, and can include a proximal end 312 and a distal end 314. In some embodiments, the tube 310 can include an open-ended channel or lumen centrally located and extending from the proximal end 312 and a distal end 314. In some embodiments, the tube 310 can be slidably nested within sheath 320 and extendable from a proximal end 322 of sheath 320. The tube 310 can include any combination of size and shapes known in the art to enable the tube 310 to slide and rotate within the lumen of the sheath 320, as discussed in greater detail herein.

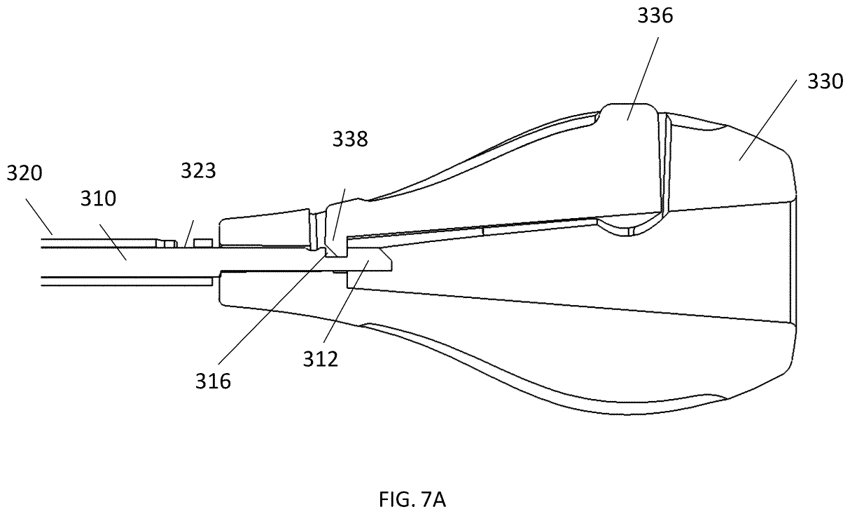

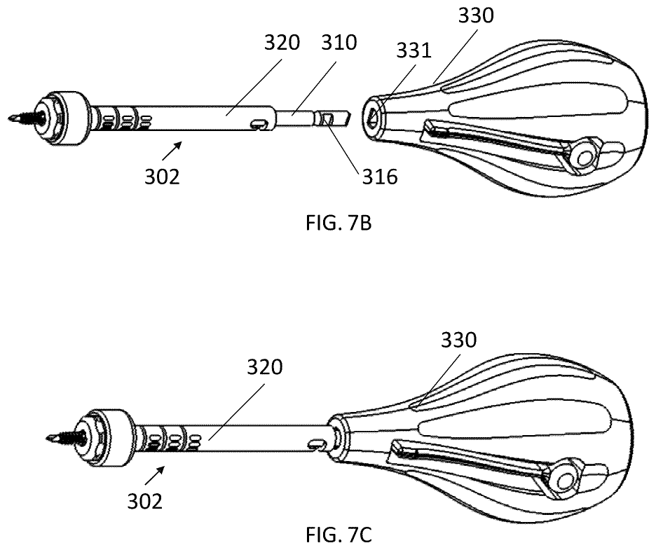

Referring to FIGS. 7A-7C, in some embodiments, the handle 330 can be designed to removably interface with the drive shaft 302 to form the driving mechanism 300. For example, the distal end 334 of the handle 330 can include an opening 331 that is sized and shaped to receive the proximal end 312 of the tube 310. In some embodiments, the combination of the tube 310 and the handle 330 can include components to couple the two together. For example, the tube 310 can include a recess 316 that can interface with a tooth, or a tab, 338 within the handle 330 to establish a friction fit. The tube 310 and handle 330 can include any combination of designs to couple to one another. For example, the tube 310 and the handle 330 can be designed to thread with one another, have a gasket to form a friction fit, share a mechanical coupling mechanism (e.g., clip, latch, etc.), etc. Once coupled together, the handle 330 can be used to assist in the maneuverability of the drive shaft 302. For example, the handle 330 can be provided with a gripping surface for rotating the drive shaft 302, e.g., a knurled surface. In some embodiments, the tube 310 and the handle 330 can be removably coupled such that the handle 330 can be selectively disengaged and removed from the drive shaft 302. The disengagement and/or release of the handle 330 from the tube 310 can include any combination of release mechanisms 336. Continuing the above example, the release mechanism 336 can include a button that when depressed will cause the tab 338 within the handle 330 to separate from the recess 316 within the tube 310, allowing the proximal end 312 of the tube 310 to be removed from inside of the handle 330, as shown in FIG. 7A. Some combination of the proximal end 312 and the release mechanism 336 can be sufficiently flexible to flex and move without breaking when force is applied while being sufficiently rigid to return to its original shape after force is no longer applied.

Referring back to FIGS. 6A and 6B, example isometric views of the drive shaft 302 are depicted at different orientations. The drive shaft 302 can be substantially cylindrical assembly that includes the sheath 320 at a distal end and the tube 310 at a proximal end. As would be appreciated by one skilled in the art, the drive shaft 302, the tube 310, and the sheath 320 can include any combination of sizes and shapes known in the art. In some embodiments, the tube 310 can be designed to slide and rotate within, and relative to, the sheath 320. The tube 310 can slide into and out of at least a portion of the sheath 320 while also being rotatable within the sheath 320. The tube 310 and the sheath 320 can be interlocked together in a manner that provides a controlled and limited range of movement in relation to one another.

In some embodiments, the sheath 320 can include a guide path 321 with one or more locking slots 323 to provide guided movement of the tube 310. The guide path 321 with the one or more locking slots 323 can extend through the sidewall of the sheath 320. The guide path 321 can be shaped to accommodate at least a portion of the tube 310. For example, the tube 310 can include a locking post 311 which extends radially outward. The locking post can be disposed within the guide path 321 in an interlocking configuration while allowing the tube 310 to traverse along the guide path 321 in a controlled manner. The one or more locking slots 323 can be designed to receive at least a portion of the locking post 311 of the tube 310 and hold the tube 310 at a given location along the sheath 320. The sheath 320 can include any number of locking slots 323 and the positions of the locking slots 323 can vary based on the length of the sheath 320 and/or tube 310. For example, there can be two locking slots 323 designating two different states of the drive shaft 302. In one example, the locking slots 323 can be uniformly spaced from one another or can have variable spacing to account for different operations of the driving mechanism (e.g., retracted state, extended state, etc.).

In some embodiments, the locking slots 323 are connected to the guide path 321 to create a continuous cutout within the sidewall of the sheath 320. The locking slots 323 can break the continuity of the guide path 321 to provide an area to deviate from the guide path 321. For example, the locking slots 323 can be created as a first substantially perpendicular turn from the substantially straight line of the guide path 321 with second substantially perpendicular turn to provide a lock position away from the guide path 321. In some embodiments, the locking slots 323 can be positioned parallel to the guide path 321 and connected via a perpendicular channel. The perpendicular channel can be shaped to allow a one or more locking slots 323, of a tube 310, to rotate from the guide path 321 into a perpendicular channel of the locking slot 323. Thereafter, the one or more locking slots 323 can enable the locking post 311 to slide in a longitudinal direction to lock the tube 310 in place. For example, the combination of the locking slots 323 and the guide path 321 can create a cane shape, as shown in FIGS. 6A and 6B. The turns within the guide path 321 and the locking slots 323 can be beveled and/or tapered to enable smooth transition of the locking post 311 of the tube 310 within the guide path 321 and the locking slots 323. For example, turns between the guide path 321 and the locking slots 323 can be curved and rounded. As the locking post 311 travels within the guide path 321, the tube 310 can telescopically move within the sheath to adjust the overall length of the driving mechanism 300. It should be appreciated that the length of driving mechanism 300 may be adjusted prior to or after device is inserted into the body.

In some embodiments, the guide path 321 can extend from or near the proximal end 322 and terminates prior to approaching the distal end 324 of the sheath 320 such that the distal end 324 of the sheath 320 is solid with no openings in the sidewalls. The guide path 321 can terminate before reaching distal end 324, thereby preventing one or more locking slots 323 (and by extension, tube 310) from coming completely out of distal end 324 of the sheath 320 and thereby separating from sheath 320. In some embodiments, the proximal end 322 of the sheath 320 can include a seal (not depicted) positioned between the inside wall of the sheath 320 and the outside wall of the tube 310. The seal can be configured to prevent blood or other fluids from flowing in any annular gap between the sheath 320 and the tube 310. The seal can include any combination of mechanisms known in the art configured to prevent fluid transfer between two moving components, such as the sheath 320 and the tube 310.

In some embodiments, the locking post 311 can be sized and shaped to fit and slide within the guide path 321 to substantially limit movement in a single direction and rotate into a locking slot 323 to hold the tube 310 in place. For example, the locking post 311 can be a substantially cylindrical in shape to provide smooth movement within the guide path 321 and the locking slots 323. The locking post 311 of the present invention are not limited to the shape and orientation and can include any combination of sizes, shapes, and orientations without departing from the scope of the present invention. In some embodiments, the locking post 311 can be located at or near proximal end 312 of the tube 310.

The locking post 311 can be configured to interlock with one or more locking slots 323 of sheath 320 to lock the tube 310 in place in relation to within the sheath 320 and to provide a given length of driving mechanism 300. For example, the one or more locking slots 323 can be configured to lock a length of tube 310 extending from sheath 320 at a given location. As would be appreciated by one skilled in the art, the locking post 311 can be located at any position along the length of the tube 310 that enables the tube 310 to lock with the one or more locking slots 323 within the sheath 320. Further, there may be more than one locking post 311 disposed on the tube 310.

Referring to FIGS. 6C-6F, cross-sectional isometric and side views of an example embodiments of drive shaft 302 are depicted. In some embodiments, the drive shaft 302 can include an insertion mechanism 340 and a pathway in which the insertion mechanism 340 can extend out of the distal end 324 of the sheath 320. The insertion mechanism 340 can include any combination of devices designed for insertion, penetration, etc. into a target area. For example, the insertion mechanism 340 can be a needle. In some embodiments, the proximal end of the insertion mechanism 340 can be coupled to the distal end 314 of the tube 310 such that movement of the tube 310 effects movement of the insertion mechanism 340 relative to the sheath 320. The insertion mechanism 340 can be coupled to the tube 310 using any combination of methods. For example, the proximal end of the insertion mechanism 340 can be mechanically fit into an opening within the distal end 314 of the tube 310, as shown in FIGS. 6C and 6D. The insertion mechanism 340 can be a solid cylindrical member. In other embodiments, the insertion mechanism 340 can be a shaft having a channel extending through at least a portion the length of the device. For example, the insertion mechanism 340 can be a hollow type needle that is capable of injecting and/or removing materials.

In some embodiments, the distal end of the insertion mechanism 340 can include a shape and structure configured for puncturing tissue and/or bone. In some embodiments, the insertion mechanism 340 can be formed from constructed from materials having sufficient axial strength for puncturing tissue and/or bone. For example, the distal end of the insertion mechanism 340 may include pointed, tapered, chiseled, etc. shaped tip configured to puncture tissues and/or bone. Although the insertion mechanism 340 can be inserted through tissue and bone without the aid of predrilling, predrilling could be performed without departing from the scope of the present invention. In some embodiments, the insertion mechanism 340 can be designed to penetrate and/or pass through another device coupled to the drive shaft 302. For example, the insertion mechanism 340 can be designed to penetrate and/or pass through an intraosseous infusion port (TOP) device 100, as discussed with respect to FIGS. 1A-4, coupled to the distal end 324 of the sheath 320. In such instances, the insertion mechanism 340 can be sufficiently sharp, rigid, and long to provide penetration and/or passage through a device and into a target site (e.g., skin). With the insertion mechanism 340 disposed therein, the driving mechanism 300 can have added functionality.

Initially, to assemble the drive shaft 302, the tube 310 can be inserted into the lumen 327 of the sheath 320 via an opening in the proximal end 322 of the sheath 320. In this setup, the locking post 311 can be aligned with the guide path 321 that extends through to the proximal end 322 of the sheath 320. In some examples, the guide path 321 may not extend to the proximal end 322, thus the sheath 320 can be formed around the tube 310 with the locking post 311 aligned with the guide path 321, for example, by welding two sheath 320 halves together. Regardless of how the components are combined, when the tube 310 is in the sheath 320 with the locking post 311 within the guide path 321, the tube 310 is enabled to freely slide longitudinally within lumen 327 of the sheath 320. When in the assembled configuration, the tube 310 can be extended or retracted within the sheath 320 to a desired length to adjust the relative depth of the insertion mechanism 340.

Continuing with FIGS. 6A-6E, example illustrations depicting actuation of driving mechanism 300 using the tube 310 and sheath 320 are shown. FIGS. 6A, 6C, and 6D show examples of the drive shaft 302 adjusted for transportation, for example, with the insertion mechanism 340 withdrawn into and protected within the sheath 320 as the tube 310 is withdrawn proximally, relative to the sheath 320. FIGS. 6B, 6E, and 6F show examples of the drive shaft 302 adjusted for advancement of the insertion mechanism 340 out of the distal end of the drive shaft 302, for example with the tube 310 being advanced distally relative to the sheath 320.

In some embodiments, the driving mechanism can be transitioned between states to provide different functionalities. As shown in FIG. 6A, to lock the tube 310 in at a first desired configuration, the tube 310 can be rotated about a central axis when the locking post 311 is aligned with a nearby locking slot 323 to allow the locking post 311 to enter and interlocks with the locking slot 323. After the rotation into the locking slot 323, the locking post 311 can be advanced within a perpendicular portion of the locking slot 323 to permit locking of the locking post 311 in place. In other words, the first portion 323a of the locking slot 323 can extend circumferentially and the second portion 323b of the locking slot 323 can extend axially to create a substantially right-angle shape. In such the first desired configuration, the tube 310 can be in a so called travel configuration where the insertion mechanism 340 is in a proximal location within the sheath 320.

Although the embodiment of the sheath 320 provided herein shows a single locking slot 323, any number of locking slots 323 can be used without departing from the scope of the present disclosure. Having multiple locking slots 323 along the axial length of the guide path 321 can provide different lengths for the drive shaft 302 and other components (e.g., insertion mechanism 340). For example, interlocking the locking post 311 with a locking slot 323 closer to proximal end 322 of sheath 320 may result in a shorter overall length, whereas interlocking the locking post 311 with a locking slot 323 closer to distal end 322 of sheath 320 results in a longer overall length. Similarly, the tube 310 can include multiple locking posts 311 which can be spaced to fit multiple locking slots 323.

In some embodiments, when locked into place, the tube 310 may be prevented from further rotation about the central axis of the sheath 320, thereby allowing an operator to translate a torque from sheath 320 to tube 310. For example, when applying a torque force to the handle 330, with tube 310 coupled thereto, the torque can be transferred from the handle 330 to the tube 310 to the sheath 320 and optionally to any device coupled to the sheath 320, e.g., the IOP device. The torque force applied to the driving mechanism 300, alone or in combination with a device (e.g., the IOP device 100), can assist in driving the insertion mechanism 340 through tissue, bone, and/or other structures. For example, the torque force can allow a user to be able to penetrate the bone with the insertion mechanism 340 and adjust a length of the drive shaft 302 to accommodate different tissue thicknesses. With a fixed length device, there can be situations where the driving mechanism is too long such that a portion of a device sticks too far outside of a patient or too short such that the device is embedded in the tissue and cannot be accessed.

Referring to FIGS. 6C and 6D, the drive shaft 302 with the tube 310 in a retracted position is depicted. When in the retracted position, the insertion mechanism 340 remains within the drive shaft 302 at a position proximal to an IOP device 100 which can be coupled to the sheath 320. In some embodiments, the interior portion of the sheath 320 can include a lumen 327 sized and shaped to allow, and limit, movement of the tube 310 therein. For example, the size and shape of the lumen 327 within the sheath 320 can assist in limiting how far the tube 310 can be advanced in one or both directions within the sheath 320. The lumen 327 within the sheath 320 can work in concert with the guide path 321, the locking slots 323, and the locking post 311 to provide controlled movement of the insertion mechanism 340.

FIGS. 6E and 6F show the drive shaft 302 with the tube 310 in an advanced position. When in the advanced position, the insertion mechanism 340 can extend distally from the drive shaft 302 and/or through any device, e.g., the IOP device 100, coupled to the sheath 320. In some embodiments, an amount of advancement of the insertion mechanism 340 out of the distal end 324 of the sheath 320 can be limited by the length and shape of the lumen 327 to limit movement of the tube 310 therein. When the driving mechanism 300 is in the advanced position, the insertion mechanism 340 can be a hollow structure that defines a continuous pathway which can extend from the distal end of the insertion mechanism 340 through the drive shaft 302 and any device (e.g., device 100) attached thereto. The continuous pathway can be sized and dimensioned to allow for access to/from an insertion site, via the insertion mechanism 340, to an opposing end of the drive shaft 302.

Referring to FIG. 7A, in some embodiments, the distal end 334 of the handle 330 can include a tab 338 that is sized and shaped to interface with a recess 316 in the proximal end 312 of the tube 310 to couple the two together. The tab 338 within the handle 330 can be pushed out of the way as the proximal end 312 of the tube 310 contacts the tab 338. For example, the tab 338 can have a bevel that will push the tab 338 upward when contacted by the proximal end 312 of the tube 310. In some embodiments, the tab 338 can be coupled to the release mechanism 336, e.g., as a two piece assembly or a unitary structure. Similar to the tube 310 pushing on the taper surface of the tab 338, the compression of the release mechanisms 336 can cause the tab 338 to lift such that the handle 330 can be selectively disengaged and removed from the proximal end 312 of the tube 310. For example, the release mechanism 336 can include a button that when depressed will cause the tab 338 within the handle 330 to separate from the recess 316 within the tube 310, allowing the proximal end 312 of the tube 310 to be removed from inside of the handle 330.

Referring to FIG. 7B, in some embodiments, regardless of coupling design, the drive shaft 302 can be inserted into an opening within the handle 330 to join the two together to create the driving mechanism 300. The handle 330 can be designed to removably couple to the proximal end of the drive shaft 302 (e.g., proximal end 312 of the tube 310). In some embodiments, the opening in the handle 330 can be sized and shaped to the same size and shape of the proximal end of the drive shaft 302. The opening within the handle 330 can also be designed such that the handle 330 and proximal end of the drive shaft 302 should be oriented in a particular alignment. For example, as depicted in FIG. 7B, the opening can be a ‘D’ shape, although any combination of shaped that can allow for a rotational force to be transferred can be used. Once the proximal end 312 of the tube 310 is inserted the handle 330, the drive shaft 302 can be fixedly attached for use as the driving mechanism 300. Referring to FIG. 7C, in some embodiments, once coupled together, the handle 330 can be used to assist in the maneuverability of the drive shaft 302. For example, the handle 330 can be provide a gripping surface for applying a rotational force to the drive shaft 302. The handle 330 can be any combination of sizes and shapes to enable a user to ergonomically hold and apply a rotational force to the drive shaft 302 and optionally the device 100 via the drive shaft 302, through tissue and into bone.

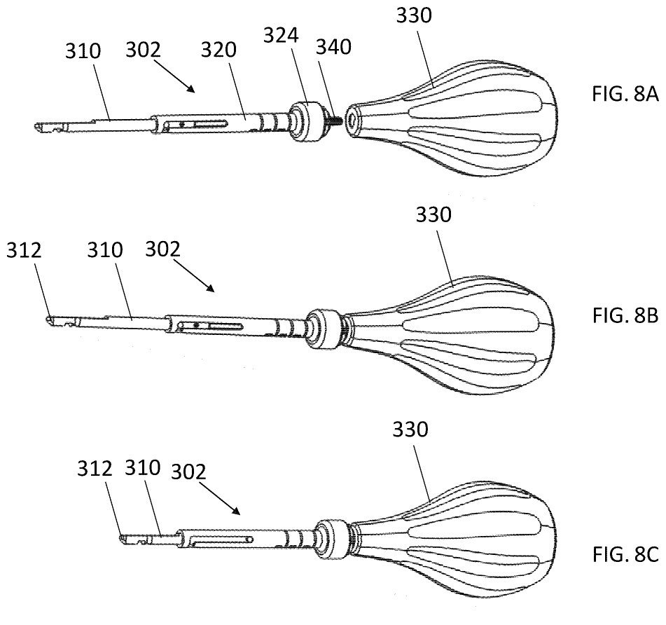

Referring to FIGS. 8A-8C, the handle 330 can be designed to removably couple to the distal end of the drive shaft 302 in an alternative arrangement of the driving mechanism. For example, the handle 330 can be designed to interface with the distal end 324 of the sheath 320 and/or a distal end of the device 100 coupled to the distal end of the sheath 320 for a particular purpose. For example, during transport, it may be advantageous for the distal end 324 of the sheath 320 to be fixed within the handle 330 to prevent the insertion mechanism 340 from piercing the packaging and/or injuring an operator during removal. Referring to FIG. 8A the distal end 324 of the sheath 320 with the device 100 coupled therein can be aligned with the opening within the handle 330. In some embodiments, as shown in FIGS. 8A and 8B, the drive shaft 302 can be in a retracted transportation state with the insertion mechanism 340 protected within the sheath 320. Referring to FIG. 8B the distal end 324 of the sheath 320 with the device 100 coupled therein can be inserted into the opening within the handle 330. Insertion into the opening of the handle 330 can be a coupled connection such that the two components are coupled together or a non-coupled connection such that the two components are held in place together by a user. Referring to FIG. 8C, in some embodiments, when at least a portion of the device 100 is inserted within the opening in the handle 330, the drive shaft 302 can be transitioned into an extended state with the tube 310 extending out of the sheath 320. In this orientation, the handle 330 can provide a point to provide push back for advancing the proximal end 312 of the tube 310 out of the sheath 320 and potentially penetrating through a membrane, septum, etc. of a device coupled to the sheath 320. Such a penetration can be performed to prepare the device for insertion, for example, creating a pilot hole. It may be preferred to pre-penetrate the membrane, septum, etc. of the device so that there is not unnecessary force applied to the device when it is positioned on a patient for insertion.

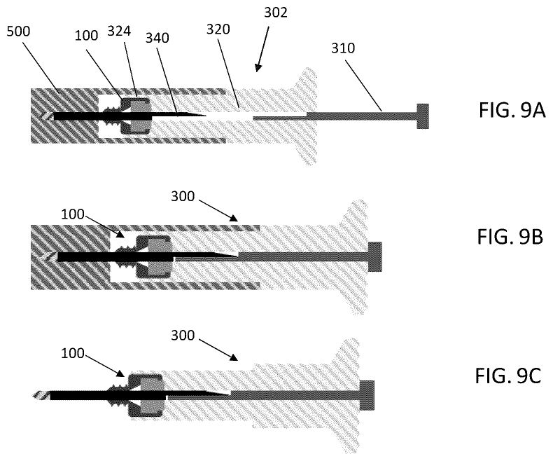

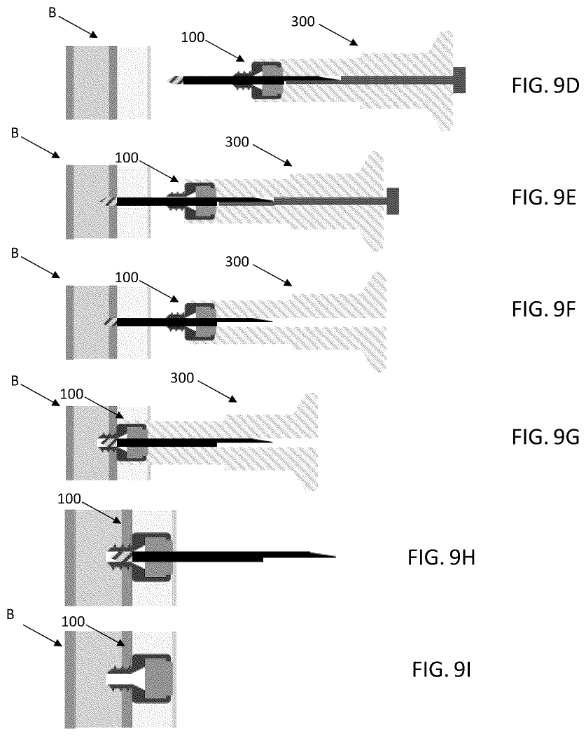

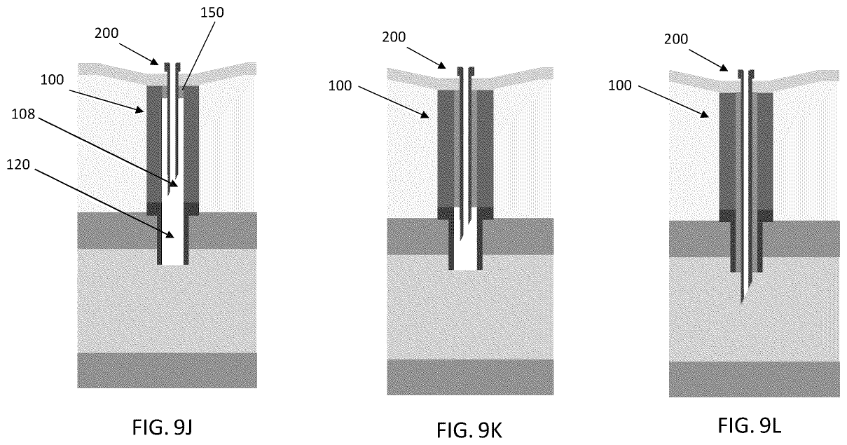

In operation, the driving mechanism 300 of the present invention can be inserted through the skin, through overlying tissue, and penetrate the bone B of a patient. Referring to FIGS. 9A-9C, the driving mechanism 300 can be packaged with an IOP device 100 in a packaging sleeve 500. As seen in FIG. 9A, the tube 310 can be disengaged from the insertion mechanism 340 for travel. The tube 310 can be advanced distally to interlock with the insertion mechanism 340, as seen in FIG. 9B, and then the driving mechanism 300 coupled to the IOP device 100 can be removed from the packaging sleeve 500 in advance of implantation of the IOP device.

Now referring to FIGS. 9D-91, once the drive shaft 302 and/or the device 100 is prepped, then they can be used for insertion into a body, leaving the IOP device 100 fixed to bone “B” while removing the driving mechanism 300. In some embodiments, the driving mechanism 300 can be inserted by first positioning the tube 310 into the sheath 320 and locking the one or more locking posts 311 into the locking slot 323. For insertion into a body, the handle 330 and the drive shaft 302 can then be reconfigured into the configuration shown in FIG. 7C.

In some embodiments, the IOP device 100 can be inserted into the distal end 324 of the sheath 320. In other examples, the IOP device 100 can be preinstalled in the distal end 324, or socket, of the sheath 320. For example, the shape of the top proximal portion 102 of the device 100 and the opening in the distal end 324 of the sheath 320 can be complimentarily to one another to form a tight fit, akin to a socket wrench. In some embodiments, the insertion mechanism 340 can extend out of the distal end of the device 100. In some embodiments, before insertion into the bone, the implantation kit (minus the handle 330) may be positioned at an insertion site, where the skin may be numbed and an incision can be created, as seen in FIG. 9D. The IOP device 100 may then be inserted into the bone B with the driving mechanism 300 to advance the IOP device 100 through the incision to reach the bone site, as seen in FIGS. 9E-9G. For example, the driving mechanism 300 may be configured to advance the IOP device 100 into a bone by applying both an axial force and a rotational torque on the IOP device 100 to drive the IOP device into the bone. During insertion, a downward and/or torque force can be applied to a handle 330, which can be transferred to the sheath 320 body and to the tube 310 via the interlocked connection of the one or more locking slots 323 in the locking slot 323. As force is applied to the tube 310, the distal tip of the insertion mechanism 340 can penetrate tissue and bone as the driving mechanism 300 is advanced.

Once the IOP device 100 is positioned at the bone site, as seen in FIG. 9E, the driving mechanism 300 may advance the IOP device 100 further into the bone by applying a rotating force to the proximal portion 102 of the device 100. In an exemplary positioning, the insertion mechanism 340 can penetrate the bone and enter bone marrow cavity. In some embodiments, the sheath 320 can be located within the overlying tissue (fat, muscle, etc.). With the driving mechanism 300 in place (e.g., with the distal tip of the insertion mechanism 340 penetrating bone into bone marrow), the continuous channel through a combination of the sheath 320, tube 310, and handle 330 can provide access for needle, or another device, to deliver fluid directly to the medullary cavity of the bone. In some examples, as seen in FIGS. 9H and 91, the driving mechanism 340 can be removed from the TOP device, while the insertion mechanism 340 can remain inserted through the IOP device 100, to provide access to the medullary cavity. The operator can then ensure that the IOP device 100 has penetrated the bone “B” to a sufficient depth to provide the requisite access to the medullary cavity and then remove the insertion mechanism 340, as seen in FIG. 91. Once the installation of the IOP device 100 is complete, the surgical site can be closed according to known methods. The design and configuration of the driving mechanism 300 can be for rapid deployment into the bone to start delivering fluid quickly.

With the IOP device 100 in place and the driving mechanism 300 removed, the device 100 may be used to infuse substances into a patient. In some embodiments, an insertion device 200, or needle, may be inserted into the device 100 to deliver fluids or medicines to and from a bone marrow cavity. The IOP device 100 may be partially or entirely submerged underneath a layer of skin covered by a septum 150 designed to prevent reflux of the infused substance into the subcutaneous tissue, as shown in FIG. 3. The insertion device 200 can penetrate through the septum 150 and into the channel 120 of the device 100. Fluids and other substances can then be delivered through the needle and into the bone marrow cavity. In some embodiment, a small volume of saline or other liquid can be infused into the channel 120 to ensure a proper positioning of the needle. In some embodiments, the septum 150 can extend partially or fully across the channel 120 of the device 100. Correspondingly, the length of the insertion device 200 can vary so the needle can reach different penetration depth within the device 100 and the bone marrow cavity. For example, the insertion device 200 can be of sufficient length such it can reach inside the channel 120 of the IOP device 100. In some embodiments, the insertion device 200 may be long enough such that a tip of the needle can go into the bone marrow cavity, as illustrated in FIGS. 9J-9L. As such, the needle can easily penetrate through material buildups or clogs within the device 100 to ensure a good flow into the marrow cavity. Moreover, for configurations where an IOP device has no hollow chambers, a needle can also go into the marrow cavity.

One advantage offered by the IOP devices presented by the present disclosure is that the IOP devices do not come into contact with free-flowing blood. As such, unlike traditional VADs, no draw back to confirm blood return through the needle is needed.

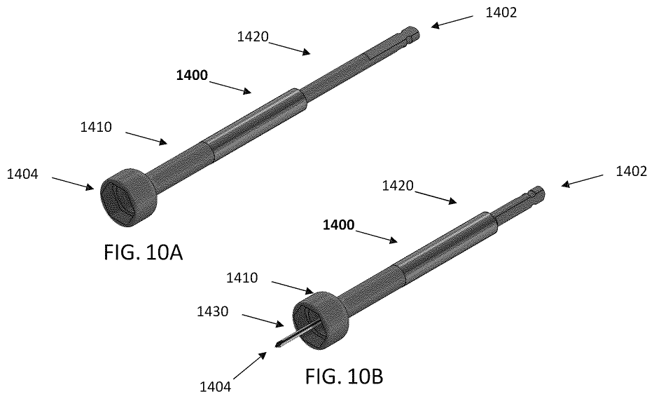

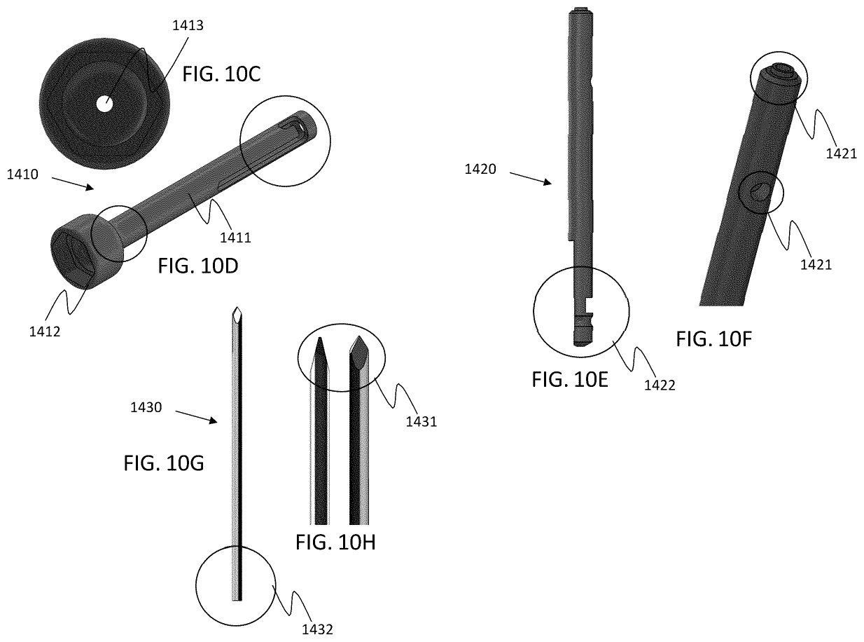

FIGS. 10A and 10B show one example of an alternative driving mechanism 1400 in extended and retracted conditions, respectively. As shown, driving mechanism 1400 can extend between a proximal end 1402 that is closer to the operator and a distal end 1404 that is closer to the patient. The driving mechanism 1400 can generally include three components: a port driver 1410 adjacent distal end 1404, a stylet driver 1420 disposed adjacent proximal end 1402, and a stylet 1430 disposed at least partially within port driver 1410 and coupled to stylet driver 1420 and disposed at least partially within port driver 1410. Each of stylet driver 1420 and stylet 1430 may be moveable or translatable relative to port driver 1410. Driving mechanism 1400 may be transitionable between a retracted condition (FIG. 10A) where stylet 1430 is fully retracted within port driver 1410, and an advanced condition (FIG. 10B) where stylet 1430 is at least partially extended from port driver 1410.

In FIGS. 10C and 10D, a port driver 1410 is shown. The port driver 1410 may include a substantially cylindrical and rigid member 1411 and a socket 1412 having an inner cavity as previously described. In some embodiments, the rigid member 1411 may include an open-ended channel or lumen centrally located and extending from the proximal end to the distal end. At least a portion of the lumen can be designed to correspond to the profile of the stylet driver 1420 as previously described. In some embodiments, socket 1412 can be sized and shaped to receive and hold a proximal portion of an IOP device and may include any combination of shapes that compliments the shape of the proximal portion of the IOP device 100 to apply torque thereto. In the example shown, port driver 1410 has an increased cross-sectional area than previous examples and includes a terminal hole 1413 configured and sized to allow a stylet to pass therethrough. Port driver 1410 may couple or mate with stylet driver 1420 (FIGS. 10E & 10F) via or at mating features 1421. In some examples, port driver 1410 and stylet driver 1420 may be welded together or connected via pins, screws, or other fasteners. Additionally, stylet driver 1420 may include one or more couplers 1422 to couple or join to a handle (not shown). The two components may be coupled together so that one can translate relative to the other (e.g., telescope) while retaining the coupling of the two components. Finally, stylet 1430 may be similar to those previously described but may include a modified geometry to reduce the length of the stylet that protrudes from the port driver. In at least some examples, the stylet 1430 may protrude out of the port driver within a range of 3-30 mm, in one example the stylet 1430 can protrude out of the port driver about 5 mm. Stylet 1430 may include a beveled tip 1431 to puncture the septum of an IOP device at one end, and a flat edge 1432 at the other end, as seen in FIGS. 10G and 10H.

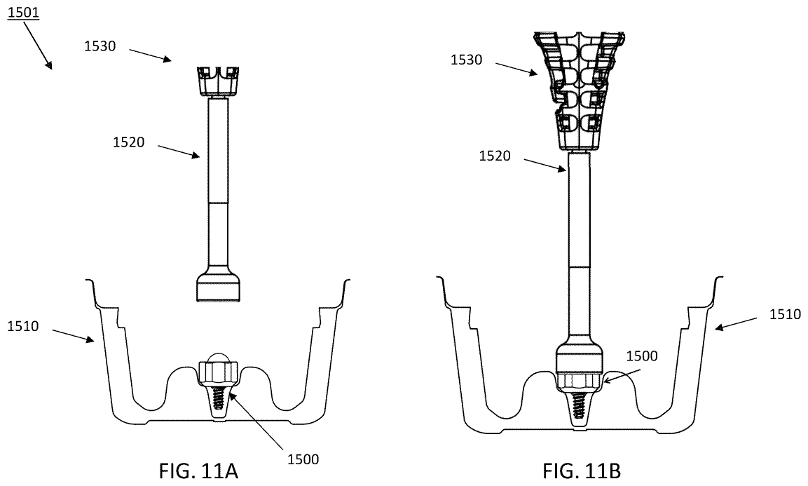

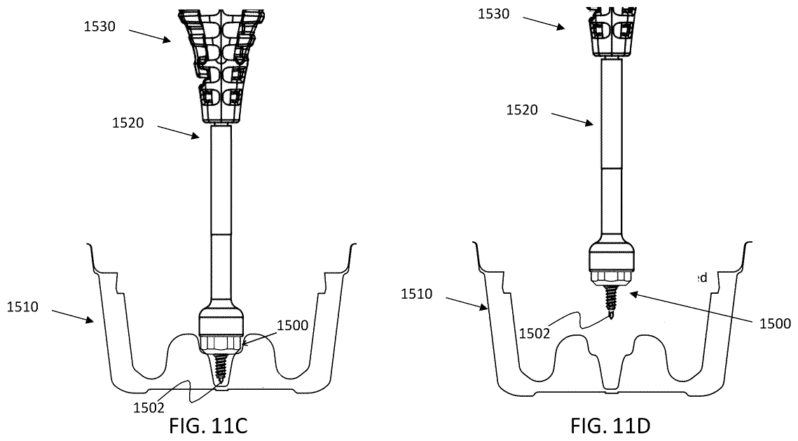

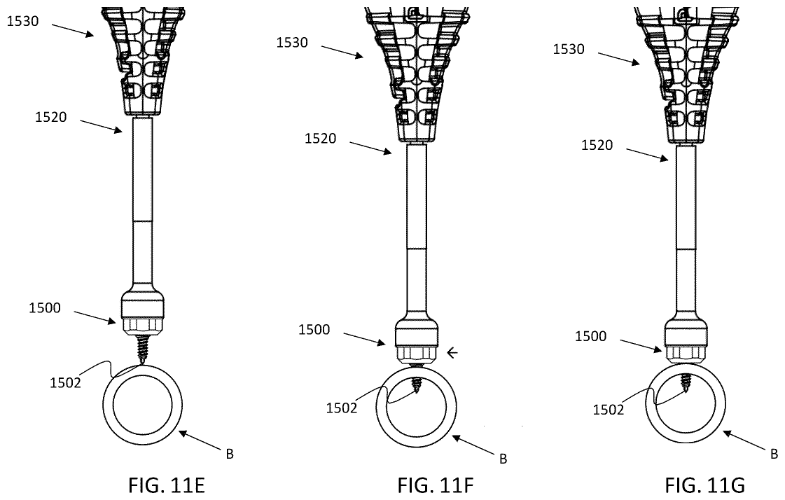

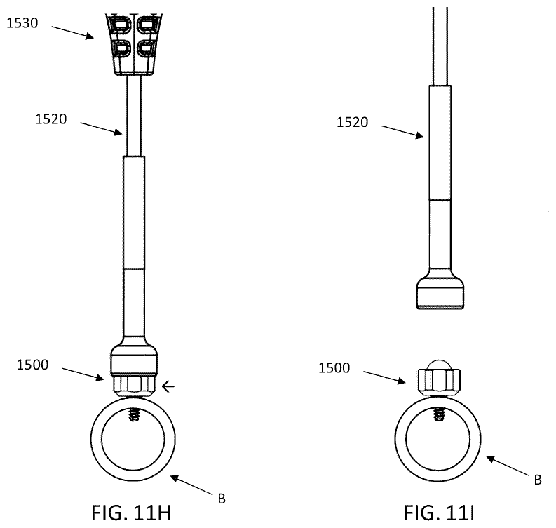

FIGS. 11A-11I are schematic cross-sectional images showing the use of a system 1501 to deliver and fix an IOP device within cortical bone. Briefly, the instant intraosseous infusion system 1501 is configured to provide an alternative route of access to facilitate fluid infusion into the circulatory system. The system 1501 can be comprised of an IOP device 1500 and an accompanying delivery/fixation system. The IOP device is configured to be screwed into a target bone cortex using the supplied delivery/fixation system. A standard hand-powered orthopedic AO driver (not shown) may also be used in the place of the supplied handle.

In this example, IOP device 1500 consists of a rigid housing made out of titanium and self-sealing silicone septum and is designed to allow delivery of fluids using a non-coring access needle (not shown). The IOP device 1500 can be any of the IOP devices 100 described above. To facilitate implantation, the stem of the TOP device can be a self-tapping bone screw and the external body of the IOP device can be a male hex-shape used to interface with the delivery system. In this example, the delivery system can be stainless steel and can contains a stainless-steel stylet to facilitate placement of the TOP device. As will be described in greater detail, an adapter can be rotated and advanced to reveal an internal stylet.

In use, the operator may first evaluate the patient and choose the most appropriate anatomic site for the TOP device placement. The patient may be positioned for appropriate exposure to the chosen access site, and using institutional protocols and adhering to institutional policy, the operator may use sterile technique to clean, prepare, and drape the insertion site and for the remainder of the procedure. Adequate local anesthesia, or other anesthesia techniques, may be provided. The operator may open the TOP device packaging 1510 using sterile technique and ensure all components are included, as shown in FIG. 11A. The operator may then identify the intended implant site and make a 1.5 to 2.5 cm skin incision slightly off-centered from the targeted implant site. Using an instrument or blunt digital dissection, the operator may clear the bone surface from soft tissue and muscle and ensure there is sufficient bone surface available for seating the TOP device 1500 into the bone.

The operator may use handle 1530 to place the driving mechanism 1520 above the TOP device 1500 (FIG. 11A) and lower the driving mechanism 1520 onto the TOP device 1500 (FIG. 11B). The operator may then hold the driving mechanism stationary, retract handle 1530 upwards, then rotate handle 1530 counterclockwise until a hard stop is felt (FIG. 11C). In this example, stylet 1502 can be advanced through the TOP device 1500 and the sharp tip can be exposed distal to the TOP device 1500. The operator may use handle 1530 to remove the TOP device 1500 from the packaging 1510 (FIG. 11D).

With the TOP device 1500 removed from the packaging, the operator may maintain a perpendicular orientation and press the assembly until the TOP device 1500 contacts the target bone surface “B” (FIG. 11E). In this example, the bone “B” is shown as illustrated as a circular element. Handle 1530 may be rotated using a smooth clockwise turning motion while applying a slight downward force. Using this technique, the user may drill the stylet 1502 into the cortex of the bone until the TOP device 1500 begins to thread into the bone (FIG. 11F). Next, using gentle forward pressure, the operator may rotate handle 1530 clockwise to screw the TOP device 1500 into the bone until the threaded portion is entirely beneath the bone surface and the bottom of the head of the device is fully seated on the surface of the target bone (FIG. 11G) to thereby stop the TOP device from being inserted any further. Once the resistance of a fully-seated TOP device 1500 is felt, the operator may stop rotating the handle. At this stage, the operator may visually inspect and verify that the TOP device 1500 base is fully seated on the surface of the target bone. If TOP device 1500 appears to be fully seated, the user may hold the driving mechanism stationary, retract the handle 1530 upwards, rotate the handle clockwise until a hard stop is felt, then advance the handle 1530 downwards until a hard stop is felt (FIG. 11H). In this position, the stylet is now retracted back inside the driving mechanism and the sharp tip is no longer exposed. If the TOP device 1500 does not appear to be fully seated, the operator may continue to screw the TOP device 1500 into the target bone as previously described. If the TOP appears to be properly seated, the driving mechanism may be removed (FIG. 11I), and the user may palpate the TOP device 1500 to verify that the bottom of the head of the device is fully seated on the surface of the target bone. If the bottom of the head of the device is not fully seated on the surface of the target bone, the operator may place the driving mechanism back onto the TOP device 1500 (in the retracted position) and continue to screw the TOP device 1500 into the target bone.

Once the bottom of the head of the device is fully seated on the surface of the target bone then, using a 19 Ga or smaller non-coring Huber needle, the operator may access TOP device 1500 in the center of the septum in a perpendicular orientation, and advance the needle into the medullary space. If the metal wall of the TOP device 1500 is felt, the operator may simply pull back on the needle and reposition slightly (adjust by −1 mm increments) towards the center and re-advance the needle. Using a syringe (5 cc or larger) or pressure infusion set (up to 300 mmHg), the operator may infuse up to 25 cc of isotonic fluid (such as normal saline, 0.9% Sodium Chloride) through the needle into the medullary space to confirm proper intramedullary position. The skin may be closed using any desired closure technique and sterile dressing may be applied, per institutional protocol. While certain measurements are provided as exemplary embodiments, other syringes, fluids, and volume of fluids, etc., can be used without departing from the spirit of this disclosure.

To use the TOP device in conjunction with needle insertion for delivery of fluid or medication, the operator may perform any or all of the following steps:
1. Palpate the TOP device to accurately locate the needle insertion site (center of the septum).
2. Clean the needle insertion site with 2% chlorohexidine and sponge applicator starting at the access site and in a spiral fashion, working outwards to at least 8 cm from the insertion site and for at least 30 seconds.
3. Open the necessary equipment/supplies onto a sterile field. Huber, non-coring needle; syringe; extension tubing, dressing, sterile barrier drape
4. Assemble the non-coring needle and extension tubing if necessary and prime/de-air with isotonic saline solution (e.g., 0.9% normal saline).
5. Drape the insertion site with sterile barrier drape.
6. Apply or administer topical or local anesthesia to the needle insertion site if desired.
7. Locate the insertion site by palpating the center of the TOP septum and digitally stabilize the device and skin.
8. In a perpendicular manner, with the needle at a 90 degree angle to the skin and IOP septum, firmly insert the non-coring needle through the skin and into the center of the IOP septum. If the metal wall of the IOP is felt, simply pull back on the needle and reposition slightly (adjust by −1 mm increments) towards the center and re-advance the needle.
9. Push the needle in until the tip of the needle is estimated to reach the medullary space.
10. Using a suitable syringe or lager, slowly inject up to 10 cc of isotonic saline solution to assess patency.
11. Once correct needle position is confirmed, apply an appropriate self-adherent dressing to the site, needle and extension tubing to maintain a sterile barrier and secure the needle in the proper position.
12. Attach the desired, de-aired infusion set to the extension tubing and administer fluid/medication as indicated.
13. Once the infusion is complete, completely close the infusion set using the attached pinch or roller clamp as appropriate.

It will be appreciated that procedures and steps described herein are merely exemplary and not to be taken as limiting. Specifically, it will be understood that any of the steps described above with respect to the loading, delivery and/or fixation of the IOP device, and/or the introduction of medicine or fluid through the IOP device may be optional and that the steps may be performed in a different order. As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. An implantable device comprising:
a proximal portion designed for subcutaneous placement, said proximal portion including a concave inlet extending distally toward a first channel;
a distal portion designed for intraosseous placement at a site of interest, said distal portion having a second channel terminating in an opening and being co-axially aligned and in fluid communication with the first channel to define a pathway from the proximal portion to the site of interest; and
a penetrable seal situated between the first channel and the second channel to minimize backflow from the site of interest into the proximal portion.

2. The implantable device of claim 1, wherein said concave inlet has a surface defined by a rounded arc which is curved towards a central axis of the pathway.

3. The implantable device of claim 1, wherein said distal portion further comprises one or more retention elements to assist the implantation and anchoring of the distal portion within bone.

4. The implantable device of claim 1, wherein the proximal portion further comprises a recess at a distal end to receive at least one of the penetrable seal and a top end of the intraosseous portion.

5. The implantable device of claim 4, wherein the proximal portion further comprises overhanging ends encasing at least one of the penetrable seal and the top of the distal intraosseous portion.

6. The implantable device of claim 1, wherein the second channel includes a tapered inlet at a proximal end.

7. The implantable device of claim 1, wherein the penetrable seal is a funnel shaped with one or more resealable flaps.

8. The implantable device of claim 7, wherein the one or more resealable flaps extend distally towards a central axis to direct an insertion device into the second channel.

9. A system comprising:
an implantable device having:
a proximal portion designed for subcutaneous placement, said proximal portion including a first channel;
a distal portion designed for intraosseous placement at a site of interest, said distal portion including a second channel terminating in an opening and which is co-axially aligned and in fluid communication with the first channel to define a pathway from the proximal portion to the site of interest; and
a penetrable seal disposed between the first channel and the second channel to minimize backflow from the site of interest into the proximal portion; and
a driving mechanism for interfacing with a top of the proximal portion for applying axial and rotational force to the implantable device.

10. The system of claim 9, wherein the driving mechanism comprises:
a sheath including a distal opening having an interior shape which is complimentary to an outer shape of the proximal portion to receive the implantable device; and
a tube insertable into a lumen of the sheath to adjust a length of the driving mechanism.

11. The system of claim 10, wherein the tube is coupled to an insertion mechanism for advancement through a distal end of the sheath.

12. The system of claim 10, wherein the tube further comprises one or more locking posts for traversing within a guide path disposed through the sheath and said one of more locking posts designed for locking in place in said guide path.

13. The system of claim 10, wherein the driving mechanism further comprises a handle having an opening to receive a proximal end of the tube for applying the rotational force.

14. The system of claim 10, wherein an insertion mechanism is axially aligned with the first and second channels such that the insertion mechanism can be advanced through the penetrable seal of the device.

15. A method of placement of a device at a site of interest, the method comprising,
providing a device having,
a proximal portion designed for placement into a soft layer, said proximal portion including a first channel;
a distal portion designed for placement in a hardened layer at a site of interest, said distal portion including a second channel terminating in an opening and which is co-axially aligned and in fluid communication with the first channel to define a pathway from the proximal portion to the site of interest; and
a penetrable seal disposed between the first channel and the second channel to minimize backflow from the site of interest into the proximal portion;
engaging the proximal portion of the device with a driving mechanism; and
applying a rotational force on the driving mechanism to rotationally advance the distal portion of the device into the hardened layer at the site of interest.

16. The method of claim 15, further comprising inserting an insertion device through the pathway of the device.

17. The method of claim 16, wherein the proximal portion includes a concave inlet extending distally toward the first channel and the insertion device is directed towards the pathway by the concave inlet.

18. The method of claim 16, wherein the insertion device is configured to deliver a substance to the site of interest or collect a substance from the site of interest.

19. The method of claim 15, wherein after the applying step, the device is later identified by palpating a center of the device.

20. The method of claim 15, wherein the driving mechanism comprises a distal opening having an interior shape which is complimentary to an outer shape of the proximal portion to receive the device.
